SEQUENCE LISTING (10 sequences)

SEQ ID NO: 1 - DNA, 1131 bp, Homo sapiens
  CDS (235)..(1128)
aaaacagaacagacccccatccctgggctggaggacccgcctcttggcagccagctgagaaggcgccccggggagggggaaactgacatcccatctagagccgtccctcctcttcctcccctcccgactctctgctcctttcccgccccagaagttcaagggcccccggcctcctgcgctcctgccgcagggaccctcgacctcctcagagcagccggctgccgccccgggaagatggcgaggaggagccgccaccgcctcctcctgctgctgctgcgctacctggtggtcgccctgggctatcataaggcctatgggttttctgccccaaaagaccaacaagtagtcacagcagtagagtaccaagaggctattttagcctgcaaaaccccaaagaagactgtttcctccagattagagtggaagaaactgggtcggagtgtctcctttgtctactatcaacagactcttcaaggtgattttaaaaatcgagctgagatgatagatttcaatatccggatcaaaaatgtgacaagaagtgatgcggggaaatatcgttgtgaagttagtgccccatctgagcaaggccaaaacctggaagaggatacagtcactctggaagtattagtggctccagcagttccatcatgtgaagtaccctcttctgctctgagtggaactgtggtagagctacgatgtcaagacaaagaagggaatccagctcctgaatacacatggtttaaggatggcatccgtttgctagaaaatcccagacttggctcccaaagcaccaacagctcatacacaatgaatacaaaaactggaactctgcaatttaatactgtttccaaactggacactggagaatattcctgtgaagcccgcaattctgttggatatcgcaggtgtcctgggaaacgaatgcaagtagatgatctcaacataagtggcatcatagcagccgtagtagttgtggccttagtgatttccgtttgtggccttggtgtatgctatgctcagaggaaaggctacttttcaaaagaaacctccttccagaagagtaattcttcatctaaagccacgacaatgagtgaaaatgatttcaagcacacaaaatcctttataatttaa

SEQ ID NO: 2 - protein, 298 aa, Homo sapiens
MARRSRHRLLLLLLRYLVVALGYHKAYGFSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQVDDLNISGIIAAVVVVALVISVCGLGVCYAQRKGYFSKETSFQKSNSSSKATTMSENDFKHTKSFII

SEQ ID NO: 3 - DNA, 34 bp, Artificial Sequence
  Primer
ccccgcatcacttcttgtcacatttttgatccgg

SEQ ID NO: 4 - DNA, 25 bp, Artificial Sequence
  Primer
ctgctctgaggaggtcgagggtccc

SEQ ID NO: 5 - DNA, 26 bp, Artificial Sequence
  Primer
gccgcggatccaagatggcgaggagg

SEQ ID NO: 6 - DNA, 32 bp, Artificial Sequence
  Primer
gctattatgccggtaccgttgagatcatctac

SEQ ID NO: 7 - DNA, 23 bp, Artificial Sequence
  Primer
taaaaatcgagctgagatgatag

SEQ ID NO: 8 - DNA, 22 bp, Artificial Sequence
  Primer
ttaaattataaaggattttgtg

SEQ ID NO: 9 - DNA, 52 bp, Artificial Sequence
  Primer
tcaggcgtagtcgggcacgtcgtaggggtaaattataaaggattttgtgtgc

SEQ ID NO: 10 - protein, 9 aa, Artificial Sequence
  HA-Tag
YPYDVPDYA

(19) United States
(12) Patent Application Publication
(10) Pub. No.: US 2003/0130166 A1
(43) Pub. Date: Jul. 10, 2003

(54) Polynucleotide encoding a human junctional adhesion protein (JAM2)

(76) Inventor: Sonia Cunningham, Houston, TX (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/192,791

(22) Filed: Jul. 10, 2002

Related U.S. Application Data
(63) Continuation-in-part of application No. 10/139,849, filed on May 7, 2002.
Application No. 10/139,849 is a continuation of application No. 09/643,929, filed on Aug. 23, 2000, now abandoned.
(60) Provisional application No. 60/150,459, filed on Aug. 24, 1999.

(51) Int. Cl. A61K 31/00; G01N 33/53; G01N 33/543; G01N 33/537

(52) U.S. Cl. 514/1; 435/7.92; 514/12

(57) ABSTRACT

The present invention relates to an isolated and purified polynucleotide encoding for a human junctional adhesion protein and methods of using said protein in assays to identify certain compounds and methods of treatment using said compounds.

### RELATED APPLICATION INFORMATION

This application in a continuation-in-part of Ser. No. 10/139,849 filed May 7, 2002, which is a continuation of Ser. No. 09/643,929, filed on Aug. 23, 2000, which claims priority from U.S. Application No. 60/150,459 filed on Aug. 24, 1999.

### TECHNICAL FIELD OF THE INVENTION

The present invention relates to molecular biology. More specifically, the present invention relates to a polynucleotide which encodes a human junctional adhesion protein, a polypeptide encoded by said polynucleotide and to recombinant vectors expressing said polypeptide. The present invention also relates to methods of using said human junctional adhesion protein in assays to identify certain compounds and methods of treatment using said compounds.

### BACKGROUND OF THE INVENTION

Cell adhesion is of prime importance for the formation and functional maintenance of multicellular organisms. Adhesion proteins can be classified as cell surface molecules that mediate intercellular bonds and/or participate in cell-substratum interactions. Their intracellular domains provide a functional link to the cytoskeleton and this appears to be important for efficient cell-cell adhesion to take place. They are expressed in characteristic spatiotemperal sequences. Different superfamilies have been described including immunoglobulin (hereinafter, “Ig”), cadherin, integrin, selectin (Aplin A E, Howe A, Alahari S K, Juliano R L, (1998) Pharmacol. Rev. 50:197-263). Adhesion proteins belonging to the immunoglobulin superfamily may operate in both a homotypic and/or heterotypic manner. The common building block is the Ig domain and the prototype is neural cell adhesion molecule (hereinafter, “NCAM”) which possesses five Ig domains. This family participates in diverse biological functions including leukocyte-endothelial cell interactions, neural crest cell migration, neurite guidance and tumor invasion.

It is well demonstrated that during inflammation members of the Ig superfamily interact with and participate in leukocyte adhesion, invasion and migration through the vessel wall (Gonzalez-Amaro R, Diaz-Gonzalez F, Sanchez-Madrid F, (1998) Drugs 56:977-88). Selectins are involved in the initial interactions (tethering/rolling) of leukocytes with activated endothelium, whereas integrins and Ig superfamily CAMs mediate the firm adhesion of these cells and their subsequent extravasation.

Tight junctions (hereinafter, “TJ”) and adherens junctions (hereinafter, “AJ”) are specialized structures that occur between apposing endothelial and epithelial cells. They form a semipermeable intercellular diffusion barrier that is both dynamic and regulated. Obviously these structures must be disrupted, or reorganized, in order to facilitate leukocyte passage from the circulation. The tight junction is the most apical component of the junctional complex. In recent years, two types of transmembrane protein, namely occludin and claudins, have been described that constitute the tight junction (Fanning A S, Mitic L L, Anderson J M, (1999) J. Am. Soc Nephrol. 10:1337-45). They possess four putative transmembrane domains and occludin itself can function as an adhesion molecule. Occludin directly interacts with ZO-1, a member of the membrane-associated guanylate kinases (Furuse M, Itoh M, Hirase T, Nagafuchi A, Yonemura S, Tsukita S, Tsukita S, (1994) J. Cell. Biol. 127:1617-26). ZO-1 provides a connection to the perijunctional cytoskeleton through its ability to associate with actin filaments (Itoh M, Nagafuchi A, Moroi S, Tsukita S, (1997) J. Cell. Biol. 138(1):181-92).

The platelet endothelial cell adhesion molecule, PECAM-1, a member of the Ig superfamily of adhesion proteins, localizes to the lateral membranes between endothelial cells (Zocchi M R, Ferrero E, Leone B E, Rovere P, Bianchi E, Toninelli E, Pardi R, (1996) Eur. J. Immunol. 26:759-67). However, it is not associated with the TJ and AJ structures (Ayalon O, Sabanai H, Lampugnani M G, Dejana E, Geiger B, (1994) J. Cell. Biol. 126(1):247-58). The crucial role of PECAM-1 in paracellular migration of leukocytes to extravascular sites has been established (Muller W A, Weigl S A, Deng X, Phillips D M, (1993) J. Exp. Med. 178:449-60). In 1998 a novel mouse junctional adhesion molecule (hereinafter, “JAM1”) was cloned and identified as an additional transmembrane protein component of the tight junction (Martin-Padura I, Lostaglio S, Schneemann M, Williams L, Romano M, Fruscella P, Panzeri C, Stoppacciaro A, Ruco L, Villa A, Simmons D, Dejana E, (1998) J. Cell. Biol. 142(1):117-27). JAM1 possesses two Ig domains, a single transmembrane and a short intracellular domain. Thus it belongs to the Ig superfamily of adhesion molecules and evidence suggests that it influences the paracellular transmigration of immune cells.

Whether the extracellular domain of JAM1 engages in heterotypic interactions with other JAM family members remains to be elucidated. Most recently, the integrin LFA-1 has been demonstrated to bind to JAM1 (Ostermann G, Weber K S, Zernecke A, Schroder A, Weber C. (2002) Nat. Immunol. 3:151-8). The ability to inhibit JAM function may allow alleviation of inflammatory diseases such as arthritis, asthma, rheumatoid arthritis, IBD and Crohns.

Tight junctions are crucial structures for maintenance of the blood-brain (hereinafter, “BBB”) and blood-retinal (hereinafter, “BRB”) barriers. In some instances it may be desirable to selectively disrupt endothelial TJs. For example disruption of the BBB may provide a method for transvascular delivery of therapeutic agents to the brain (Muldoon L L, Pagel M A, Kroll R A, Roman-Goldstein S, Jones R S, Neuwelt E A, (1999) Am. J. Neuroradiol. 20:217-22). In another instance, strategies designed to open the tight junctions of polarized epithelial cells may improve gene delivery for diseases such as cystic fibrosis: here the polarized apical membranes of airway epithelial cells are resistant to transfection by lipid:pDNA complexes (Chu Q, Tousignant J D, Fang S, Jiang C, Chen L H, Cheng S H, Scheule R K, Eastman S J, (1999) Hum. Gene. Ther. 10:25-36).

Integrins are heterodimeric adhesion molecules that are involved in a variety of adhesive processes including cell to matrix attachment, and cell to cell adhesion. Integrins play key roles during normal cellular processes and contribute significantly to diseases involving inflammation and during tumor metastasis. Integrins consist of non-covalently associated α and β sub-units that combine to form specific pairs with defined ligand partners. The α4 subunit can associate with β1 to form α4β1 and with β7 to form α4β7. The α4β1 integrin interacts with the IgSF adhesion molecule VCAM in addition to the extracellular matrix molecule fibronectin. The α4β7 additionally binds MAdCAM. Ligand binding of integrins is modulated by cations.

The α4 integrins are expressed in monocytes, eosinophils and lymphocytes and their affinity and/or avidity can be up regulated following chemokine stimulation. The VCAM and MAdCAM ligands are endothelial expressed molecules that upon engagement with integrin promote leukocyte/endothelial adhesion and subsequent diapedesis. These interactions have been described in numerous pathological processes including but not limited to autoimmune diseases such as diabetes melitus, multiple sclerosis, rheumatoid arthritis (RA), systemic lupus erythematosus, Sjogren's syndrome, encephalomyelitis, in addition to chronic inflammatory diseases, such as asthma, psoriasis, transplant-rejection, inflammatory bowel disease and allergy.

### SUMMARY OF THE INVENTION

The present invention relates to an isolated and purified human JAM2 polynucleotide encoding a human JAM2 polypeptide or fragment thereof. Moreover, the present invention further relates to an isolated and purified polynucleotide having the nucleotide sequence of SEQ ID NO: 1.

The present invention also relates to an isolated and purified human JAM2 polypeptide or fragment thereof. Moreover, the present invention relates to an isolated and purified polypeptide having the amino acid sequence of SEQ ID NO: 2.

The present invention also relates to a recombinant vector. This vector contains a polynucleotide having the nucleotide sequence of SEQ ID NO: 1, which encodes for a human junctional adhesion protein. The polynucleotide is operatively linked to a promoter that controls expression of the nucleotide sequence and a termination segment.

The present invention also relates to a host cell containing the recombinant vector. The host cell can be a bacterial cell, an animal cell or a plant cell. The present invention also relates to a transgenic mammal containing the recombinant vector described herein.

The present invention further relates to an antibody, which binds to the hereinbefore described polypeptide.

In yet still a further embodiment, the present invention relates to a method of identifying a compound that modulates binding between an α4β1 or α4β7 integrin and JAM2. The method involves contacting α4β1 or α4β7 integrin and JAM2 in the presence and absence of a test compound, detecting binding between the α4β1 or α4β7 integrin and JAM2, and identifying whether the compound modulates the binding between the α4β1 or α4β7 integrin and JAM2 in view of decreased or increased binding between the α4β or α4β7 integrin and JAM2 in the presence of the compound as compared to binding in the absence of the compound. More specifically, this method can be conducted by immobilizing an α4β1 or α4β7 integrin or JAM2 or a fusion protein or fragment thereof on a solid support (“The immobilized binding partner”), labeling the α4β7 or α4β7 integrin or JAM2 not immobilized on the solid support (“the non-immobilized binding partner”) with a detectable agent, contacting the immobilized binding partner with the labeled non-immobilized binding partner in the presence and absence of a compound capable of specifically reacting with α4β1 or α4β7 integrin or JAM2 and optionally in the presence of JAM3, detecting binding between the immobilized binding partner and the non-immobilized binding partner and identifying compounds that affect binding (increase or decrease the binding) between the immobilized binding partner and the labeled non-immobilized binding partner.

In yet still a further embodiment, the present invention relates to a method of inhibiting JAM2-α4β1 or α4β7 integrin mediated interactions in a mammal. The method involves the step of administering to a mammal an effective amount of soluble JAM3 or soluble JAM2 to inhibit said interaction.

In yet still a further embodiment, the present invention relates to a method of inhibiting JAM2-α4β1 or α4β7 integrin mediated interactions in in vitro. The method involves the step of administering to an effective amount of soluble JAM3 or soluble JAM2 to the reaction mixture to inhibit said interaction.

In yet still a further embodiment, the present invention involves a method of preventing JAM2 mediated interaction with an alpha4 integrin in a mammal. The method involves administering to a mammal an effective amount of a compound identified previously to prevent said binding.

In yet still a further embodiment, the present invention involves a method of preventing JAM2 mediated interaction with an α4β1 or α4β7 integrin in a reaction mixture in vitro. The method involves administering to the reaction mixture an effective amount of a compound identified previously to prevent said binding.

### BRIEF DESCRIPTION OF THE FIGURES

FIG. 1A shows the alignment of an homologous Expressed Sequence Tag (hereinafter referred to as “EST”) obtained from the databases accessed through the home page of the National Center for Biotechnology Information at www.ncbi.nlm.nih.gov, with the open reading frame of mouse junctional adhesion protein (hereinafter referred to as “mouse JAM”). FIG. 1B shows the alignment of an overlapping EST that encodes the 3′ end of a human functional adhesion protein (hereinafter “human JAM2”) including the stop codon. Identity is shown on the DNA level. FIG. 1C summarizes the Rapid Amplification of cDNA Ends (hereinafter referred to as “RACE”) procedure employed to obtain the full open reading frame of human JAM2. The longest clones identified from each reaction are aligned with mouse JAM.

FIG. 2 shows the full cDNA and amino acid sequence for the open reading frame (ORF) of human JAM2. The predicted signal sequence and transmembrane domain are underlined. N-linked glycosylation sites are highlighted, as are cysteine residues, which form disulfide bonds within the immunoglobulin-like folds in the extracellular domain. A PKC phosphorylation site is highlighted in the intracellular domain.

FIG. 3 shows the alignment of human JAM2 (top) and mouse JAM (bottom) open reading frames. Conserved cysteine residues predicted to form disulfide bonds are bolded. Conserved PKC phosphorylation sites are single underlined.

FIG. 4 shows identification of the human JAM2 transcript on normalized multiple tissue Northern blots probed under high stringency. Transcripts were viewed by hybridization to human JAM2, actin or GAPDH [a²P]dCTP labeled probes. FIG. 4A JAM2 (i) and actin (ii) probes: peripheral blood leukocytes (lane 1); lung (lane 2); placenta (lane 3); small intestine (lane 4); liver (lane 5); kidney (lane 6); spleen (lane 7); thymus (lane 8); colon (lane 9); skeletal muscle (lane 10); heart (lane 11); brain (lane 12). FIG. 4B shows JAM2 (i) and GAPDH (ii) probes: right ventricle (lane 1); left ventricle (lane 2); right atrium (lane 3); left atrium (lane 4); apex (lane 5); aorta (lane 6); adult heart (lane 7); fetal heart (lane 8). The arrows indicate the human JAM2 transcripts.

FIG. 5 shows a Western Blot Analysis of JAM2. Cell lysis from control (lane 1) of JAM2 expressing CHO cells (lane 2) was probed with mouse polyclonal anti-JAM2 extracellular domain antibody. HSB cell lysis probed with either preimmune (lane 3) or anti-JAM2 (lane 4) antibody. Equivalent amounts of protein were loaded in all lanes.

FIG. 6 shows the localization of JAM2 expressed in Chinese Hamster Ovary cells by immunofluorescence. Stable cell lines expressing full-length JAM2 (A) or control (B) were fixed with paraformaldehyde, stained with 1:100 dilution of primary mouse anti-JAM2 antibody followed by GAM-FITC. Single angle view of cellular staining volumetrically reconstructed from 26×0.4 um z-axis planes. Working magnification, x 400. Digital contrast levels were not changed during image capture. Scale bar, 20 μm.

FIG. 7 shows screening for JAM2 counter-receptors on various leukocyte cell lines. Calcein loaded cells were added to JAM2-Fc captured in 96 well plates. Binding was performed in TBS+Ca/Mg/Mn (n=6); Wells were washed, retained cells lysed and fluorescence quantitated with a fluorimeter at excitation 485/emission 530 nm. Data from a representative experiment. Average±SEM. FU, arbitrary fluorescence units.

FIG. 8 shows cation dependence of JAM2 adhesion. Binding of HSB cells performed in TBS+Ca/Mg/Mn, BB (n—10); TBS (n=7); TBS+EDTA (n=5); TBS+Ca (n=4); TBS+Mg (n=4); TBS+Mn (n=10). Averaged data from (n) independent experiments expressed as % Binding Buffer (BB)±SEM. FU, arbitrary fluorescence units. Pairwise comparisons, by Fisher's PLSD post-hoc test, significantly different from TBS: *p<0.0001, from TBS+Ca: ^{±}p<0.0001 and from TBS+Mg: ^{±}p<0.001.

FIG. 9 shows the effects of cations on manganese stimulated JAM2 adhesion. Binding of HSB cells performed in TBS+Ca/Mg/Mn (BB); TBS+Mn; TBS+Mn/Mg; TBS+Mn/Ca. Averaged data from seven (7) independent experiments expressed as % Binding Buffer (BB)±SEM. FU, arbitrary fluorescence units. Pairwise, comparisons, by Fisher's PLSK post-hoc test, significantly different from TBS: ^{*}p<0.0001; ^{¶}p<0.01. Significantly different from TBS+Mn ^{∥}p<0.001. Significantly different from TBS+Mn/Mg: ^{#}p<0.01.

FIG. 10 shows the adhesion of JAM2 Ig domains to HSB cells. Secreted fusion proteins of JAM2 Ig fold 1 (D1), Ig fold 2 (D2) and Ig folds 1+2 (D1+2), were immobilized on ELIZA wells by a 96 well plate by capture with GAM from the media of infected SF21 cells. Average±SEM. N=6. Binding of calcein labeled HSB cells was performed in TBS (n=6) and TBS+Mn (n=6). Data expressed as average of cell number bound±SEM.

FIG. 11 shows the precipitation of surface biotinylated proteins from HSB cells. Plasma membranes of K562 (lane 1) and HSB (lanes 2, 3) cells were surface biotinylated and specific binding proteins precipitated with either JAM2-Fc (lanes 1, 2) or JAM1-Fc (lane 3). JAM1 is the human homologue of mouse JAM, Genbank ACC No. U89915. Bands were viewed with avidin-HRP and ECL following electrophoresis and transfer. Equivalent amounts of protein were loaded in all lanes.

FIG. 12A shows the attenuation of JAM2 binding to an α4 integrin. JAM2 adhesion to HSB cells was assessed in TBS and TBS+Mn. The effects of the selective α4 integrin inhibitor, TBC 772 as compared to the control scrambled peptide TBC 1194. Data expressed as average of cell number bound±SEM (n=6). FIG. 12B shows a dose-response for inhibition of JAM2 adhesion to α4 integrin. JAM2 adhesion to HSB cells was assessed in TBS ( ) and TBS+Mn ( ) under identical conditions to those described for FIG. 12A.

FIG. 13 shows the ability of soluble JAM3 or neutralizing JAM3 antibodies to prevent not only JAM2 adhesion to JAM3, but also JAM2 adhesion to the α4 integrin. JAM2 adhesion to HSB cells was assessed in TBS and TBS+Mn. Additions were either: soluble JAM3 at a 10-fold molar ratio (13A) or a 1:500 dilution of neutralizing JAM3 antiserum (13B). Data expressed as average of cell number bound±SEM (n=6).

FIG. 14 shows the specific inhibition of JAM2 to integrin using neutralizing antibodies against the α4 integrin and the β1 integrin. JAM2 adhesion to HSB cells was assessed in TBS and TBS+Mn. Neutralizing antibodies (+); isotype controls (−) were preincubated with cells for 30 min prior to addition of cells to JAM2 coated 96 wells. Data expressed as average of cell number bound±SEM (n=6).

FIG. 15 shows the amino acid sequence of Human Junctional Adhesion Molecule 3 (hereinafter “JAM3”) polypeptide and its alignment with human JAM2 polypeptide and human JAM1 polypeptide. The sequence was highlighted using BOXSHADE; (▾)=predicted cleavage of signal sequence; (*), conserved cysteine residues; (•), N-linked glycosylation sites; (______); (♦), PKC phosphorylation site; (------), PDZ binding domain.

### DETAILED DESCRIPTION OF THE INVENTION

I. The Present Invention

The present invention relates to an isolated and purified polynucleotide sequence, which encodes for a human junctional adhesion protein (referred to herein as “human JAM2”). In another embodiment, the present invention relates to polypeptides for human JAM 2. In yet another embodiment, the present invention relates to recombinant vectors, which, upon expression, produce human JAM2. The present invention also relates to host cells transformed with these recombinant vectors.

II. Sequence Listing

The present application also contains a sequence listing that contains 9 sequences. The sequence listing contains nucleotide sequences and amino acid sequences. For the nucleotide sequences, the base pairs are represented by the following base codes:

| Symbol | Meaning |
|---|---|
| A | A; adenine |
| C | C; cytosine |
| G | G; guanine |
| T | T; thymine |
| U | U; uracil |
| M | A or C |
| R | A or G |
| W | A or T/U |
| S | C or G |
| Y | C or T/U |
| K | G or T/U |
| V | A or C or G; not T/U |
| H | A or C or T/U; not G |
| D | A or G or T/U; not C |
| B | C or G or T/U; not A |
| N | (A or C or G or T/U) |

The amino acids shown in the application are in the L-form and are represented by the following amino acid-three letter abbreviations:

| Abbreviation | Amino acid name |
|---|---|
| Ala | L-Alanine |
| Arg | L-Arginine |
| Asn | L-Asparagine |
| Asp | L-Aspartic Acid |
| Asx | L-Aspartic Acid or Asparagine |
| Cys | L-Cysteine |
| Glu | L-Glutamic Acid |
| Gln | L-Glutamine |
| Glx | L-Glutamine or Glutamic Acid |
| Gly | L-Glycine |
| His | L-Histidine |
| Ile | L-Isoleucine |
| Leu | L-Leucine |
| Lys | L-Lysine |
| Met | L-Methionine |
| Phe | L-Phenylalanine |
| Pro | L-Proline |
| Ser | L-Serine |
| Thr | L-Threoine |
| Trp | L-Tryptophan |
| Tyr | L-Tyrosine |
| Val | L-Valine |
| Xaa | L-Unknown or other |

III. Polynucleotides

In one aspect, the present invention provides an isolated and purified polynucleotide, which encodes human JAM2. This polynucleotide can be a DNA molecule, such as a gene sequence, cDNA or synthetic DNA. The DNA molecule can be double-stranded or single-stranded, and if single stranded, may be the coding strand. In addition, the polynucleotide can be RNA molecules such as mRNAs.

The present invention also provides non-coding strands (anti-sense) which are complementary to the coding sequences as well as RNA sequences identical to or complementary to those coding sequences. One of ordinary skill in the art will readily appreciate that corresponding RNA sequences contain uracil (U) in place of thymidine (T).

In one embodiment, the polynucleotide of the present invention is an isolated and purified cDNA molecule that contains the coding sequence of human JAM2. An exemplary cDNA molecule is shown as SEQ ID NO: 1.

As is well known in the art, because of the degeneracy of the genetic code, there are numerous other DNA and RNA molecules that can code for the same polypeptides as those encoded by SEQ ID NO: 1 or portions or fragments thereof. The present invention also contemplates homologous polynucleotides having at least 70% homology to the sequence shown in SEQ ID NO: 1, preferably at least 80% homology, and most preferably at least 90% homology. The term “homology”, as used herein, refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of nonspecific binding may be tested by the use of a second target sequence which lacks even a partial degree of complementarity (e.g., less than about 30% identity); in the absence of nonspecific binding, the probe will not hybridize to the second non-complementary target sequence. Moreover, the present invention also contemplates naturally occurring allelic variations and mutations of the cDNA sequence set forth above so long as those variations and mutations code, on expression, for the human junctional adhesion protein. The present invention also encompasses splice variations of the JAM2 polynucleotide.

The polynucleotide of the present invention can be used in marker-aided selection using techniques that are well known in the art. Marker-aided selection does not require the complete sequence of the gene. Instead, partial sequences can be used as hybridization probes or as the basis for oligonucleotide primers to amplify by PCR or other methods to identify nucleotides specific for junctional adhesion proteins in other mammals.

IV. Polypeptides

The present invention also provides for human JAM2 polypeptides. The amino acid sequence for human JAM2 is provided in SEQ ID NO:2 and contains 298 amino acid residues.

The present invention also contemplates amino acid sequences that are substantially duplicative of the sequences set forth herein such that those sequences demonstrate like biological activity to the disclosed sequences. Such contemplated sequences include those sequences characterized by a minimal change in amino acid sequence or type (e.g., conservatively substituted sequences) which insubstantial change does not alter the basic nature and biological activity of the polypeptides.

It is well known in the art that modifications and changes can be made in the structure of a polypeptide without substantially altering the biological function of that peptide. For example, certain amino acids can be substituted for other amino acids in a given polypeptide without any appreciable loss of function. In making such changes, substitutions of like amino acid residues can be made on the basis of relative similarity of side-chain substitutents, for example, their size, charge, hydrophobicity, hydrophilicity, and the like.

As detailed in U.S. Pat. No. 4,554,101, incorporated herein by reference, the following hydrophilicity values have been assigned to amino acid residues: Arg (+3.0); Lys (+3.0); Asp (+3.0); Glu (+3.0); Ser (+0.3); Asn (+0.2); Gln (+0.2); Gly (O); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Tip (−3.4). It is understood that an amino acid residue can be substituted for another having a similar hydrophilicity value (e.g., within a value of plus or minus 2.0) and still obtain a biologically equivalent polypeptide.

In a similar manner, substitutions can be made on the basis of similarity in hydropathic index. Each amino acid residue has been assigned a hydropathic index on the basis of its hydrophobicity and charge characteristics. Those hydropathic index values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Tip (−0.9); Tyr (−1.3); Pro (−1.6); His (−3.2); Glu (−3.5); Gln (−3.5); Asp (−3.5); Asn (−3.5); Lys (−3.9); and Arg (−4.5). In making a substitution based on the hydropathic index, a value of within plus or minus 2.0 is preferred.

The polypeptides of the present invention can be chemically synthesized using standard methods known in the art, preferably solid state methods, such as the methods of Merrifield (J. Am. Chem. Soc., 85:2149-2154 (1963)). Alternatively, the proteins of the present invention can be produced using methods of DNA recombinant technology (Sambrook et al., in “Molecular Cloning—A Laboratory Manual”, 2^{ND}. Ed., Cold Spring Harbor Laboratory (1989)).

V. Recombinant Vectors

The present invention also relates to recombinant vectors that contain the polynucleotide of the present invention, host cells, which are genetically engineered with recombinant vectors of the present invention and the production of the polypeptide of the present invention by recombinant techniques.

The polynucleotide of the present invention can be employed for producing polypeptides using recombinant techniques that are well known in the art. For example, the polynucleotide may be included in any one of a variety of expression vectors for expressing a polypeptide. Such vectors include chromosomal, nonchromosomal and synthetic DNA sequences, e.g., derivatives of SV40, bacterial plasmids, phage DNA, baculovirus, yeast plasmids, vectors derived from combinations of plasmids and phage DNA, viral DNA such as vaccinia, adenovirus, fowl pox virus, and pseudorabies. One of the most popular vectors for obtaining genetic elements is from the well known cloning vector pBR322 (available from the American Type Culture Collection, Manassas, Va. as ATCC Accession Number 37017). The pBR322 “backbone” sections can be combined with an appropriate promoter and the structural sequence to be expressed. However, any other vector may be used as long as it is replicable and viable in the host.

The polynucleotide sequence of the present invention may be inserted into one of the hereinbefore-mentioned recombinant vectors, in a forward or reverse orientation. A variety of procedures, which are well known in the art may be used to achieve this. In general, the polynucleotide is inserted into an appropriate restriction endonuclease site(s).

When inserted into an appropriate expression vector, the polynucleotide of the present invention is operatively linked to an appropriate expression control sequence(s), such as a promoter, to direct mRNA synthesis. As used herein, the term “operatively linked” includes reference to a functional linkage between a promoter and a second sequence, wherein the promoter sequence initiates and mediates transcription of the DNA sequence corresponding to the second sequence. Generally, operably linked means that the nucleotide sequences being linked are contiguous and, where necessary to join two protein coding regions, contiguous and in the same reading frame. The heterologous structural sequence can encode a fusion protein including either an N-terminal or C-terminal identification peptide imparting desired characteristics, such as stablization or simplified purification of expressed recombinant product.

Promoter regions can be selected from any desired gene using chloramphenicol transferase (CAT) vectors or other vectors with selectable markers. Such promoters can be derived from operons encoding glycolytic enzymes such as 3-phosphoglycerate kinase (PGK), α-factor, acid phosphatase, or heat shock proteins. Examples of bacterial promoters which can be used include, but are not limited to, lacI, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Examples of other promoters that can be used include the polyhedrin promoter of baculovirus.

Typically, recombinant expression vectors contain an origin of replication to ensure maintenance of the vector. They preferably contain one or more selectable marker genes to provide a phenotypic trait for selection of transformed host cells. Examples of selectable marker genes which can be used include, but are not limited to, dihydrofolate reductase or neomycin resistance for eukaryotic cell culture, tetracycline or ampicillin resistance for E. coli. and the TRP1 gene for S. cerevisiae. The expression vector may also contain a ribosome binding site for translation initiation and a transcription termination segment. The vector may also include appropriate sequences for amplifying expression.

Appropriate cloning and expression vectors for use with prokaryotic and eukaryotic hosts are described in Sambrook, et al., Molecular Cloning: A Laboratory Manual, Second Edition, Cold Spring Harbor, N.Y., (1989), which is herein incorporated by reference. Large numbers of suitable vectors and promoters are commercially available and can be used in the present invention. Examples of vectors which can be used include, but are not limited to: Bacterial: pQE70, pQE60, pQE-9 (Qiagen), pBS, pD10, phagescript, psiX174, pBluescript SK, pSKS, pNH8A, pkrH16a, pNH18A, pNH46A (Stratagene); ptrc99a, PKK223-3, pKK233⁻³, pDR540, pRIT5 (Pharmacia); pGEM (Promega). Eukaryotic: pWLNEO, pSV2CAT, p) G44, pXT1, pSG (Stratagene), pSVK3, pBPV, pMSG, pSVL (Pharmacia).

In another embodiment, the present invention relates to host cells containing the hereinbefore-described recombinant vectors. The vector (such as a cloning or expression vector) containing the hereinbefore-described polynucleotide, may be employed to transform, transduce or transfect an appropriate host to permit the host to express the protein. Appropriate hosts which can be used in the present invention, include, but are not limited to prokaryotic cells such as E. coli, Streptomyces, Bacillus subtilis, Salmonella typhimurium, as well as various species within the general Pseudomonas, Streptomyces, and Staphylococcus. Lower eukaryotic cells such as yeast and insect cells such as Drosophila S2 and Spodoptera Sf9. Introduction of the recombinant construct into the host cell can be effected by calcium phosphate transfection, DEAE-Dextran mediated transfection, or electroporation (see, Davis, L., Dibner, M., Battey, L. Basic Methods in Molecular Biology, (1986), herein incorporated by reference).

Various higher eukaryotic cells such as mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 293, 3T3, CHO, HeLa and BHK cell lines. Mammalian expression vectors will contain an origin of replication, a suitable promoter and enhancer, and any necessary ribosome binding sites, polyadenylation site, splice donor and acceptor sites, transcriptional termination sequences, and 5′ flanking non-transcribed sequences. DNA sequences derived from the SV40 splice, and polyadenylation sites may be used to provide the required non-transcribed genetic elements.

The engineered host cells can be cultured in conventional nutrient media modified as appropriate for activating promoters, selecting transformants or amplifying genes encoding for the human junctional adhesion protein of the present invention. The culture conditions, such as temperature, pH and the like, are those previously used with the host cell selected for expression and can be determined experimentally, using techniques which are well known in the art.

Transcription of the polynucleotide encoding the polypeptides of the present invention by higher eukaryotes can be increased by inserting an enhancer sequence into the vector. Enhancers are cis-acting elements of DNA which are about from 10 to about 300 base pairs in length, which act on a promoter to increase its transcription. Examples of suitable enhancers which can be used in the present invention include the SV40 enhancer on the late side of the replication origin base pairs 100 to 270, a cytomegalovirus early promoter enhancer, the polyoma enhancer on the late side of the replication origin, and adenovirus enhancers.

Following transformation of a suitable host strain and growth of the host strain to an appropriate cell density, the selected promoter is induced by appropriate means (such as temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents. Such methods are well-known to those skilled in the art.

The polypeptides of the present invention can be recovered and purified from recombinant cell cultures, the cell mass or otherwise according to methods of protein chemistry which are known in the art. For example, ammonium sulfate or ethanol precipitation, acid extraction, and various forms of chromatography e.g. anion/cation exchange, phosphocellulose, hydrophobic interaction, affinity chromatography including immunoaffinity, lectin and hydroxylapatite chromatography. Other methods may include dialysis, ultrafiltration, gelfiltration, SDS-PAGE and isoelectric focusing. Protein refolding steps can be used, as necessary, in completing configuration of the mature protein. Finally, high performance liquid chromatography (hereinafter, “HPLC”) on normal or reverse systems or the like, can be employed for final purification steps.

Cell-free translation systems can also be employed to produce such polypeptides using RNAs derived from the DNA constructs of the present invention.

The cDNA sequence can be used to prepare stable cell lines expressing either wildtype JAM2 or JAM2 mutated at pertinent positions to determine which part of the molecule is responsible for function. Stable or transient cell lines can be created with JAM2 possessing a tag at either the 5′ or 3′ end, e.g. HA or Myc epitope, to enable monitoring of JAM2 function/modification/cellular interactions. Additionally, cell lines expressing recombinant JAM2, can be used to screen for small molecule inhibitors of JAM2 function.

The extracellular sequence of JAM2 can also be used to make recombinant protein fused to the Fc region of mouse/human IgG. This recombinant fusion protein can be used:

a) To screen for a JAM2 ligand. Briefly, JAM2-Fe fusion can be captured onto the surface of 96 well plates. Cultured cells e.g. monocytes can be labeled with calcien dye, incubated with the immobilized JAM2-Fc, washed and fluorescence monitored. Alternatively, the JAM2-Fc can be coupled to a solid support and then used to prepare a column for purification of solubilized proteins derived from various cells/tissues. Peptide sequencing could then identify the ligand. Another approach would be to bind the JAM2-Fc to cell lysates and perform cross-linking with DSS.

b) Upon identification of a JAM2 ligand, the JAM2-Fc can be used to screen for a small molecule inhibitor of JAM2 heterotypic interactions.

c) As a tool to neutralize JAM2 function, either heterotypic or homotypic interactions. The JAM2-Fc may be administered in vivo in various animal models in order to perturb JAM2 function. Alternatively, proof of concept studies may be conducted in vitro.

If it is discovered that JAM2 binds in a homotypic manner, recombinant protein derived from the extracellular domain can be used to analyze such interactions. Protein may not possess an Fe Tag. Single immunoglobulin-like domains can be made to determine which one is responsible for homotypic interactions. Such recombinant protein can be used to assess its ability to decrease paracellular permeability in cells expressing native or recombinant JAM2. The interactions of the separate domains with each other or with a recombinant form possessing both Ig-like domains may be assessed by various means. Examples are cross-linking with DSS, analytical ultracentrifugation or sizing columns.

The JAM2 polynucleotide can be used to identify antisense oligonucleotides for inhibition of JAM2 function in cell systems. Further, degenerate oligonucleotides may be designed to aid in the identification of additional members of this family by the polymerise chain reaction. Alternatively, low stringency hybridization of cDNA libraries may be performed with JAM2 sequence to identify closely related sequences.

The intracellular domain of JAM2 can be used to “fish” for novel interacting partners in the yeast two-hybrid system. Further, JAM2 sequence may be used to inactivate an endogenous gene by homologous recombination and thereby create a JAM2 deficient cell, tissue or animal. Such cells, tissue or animals may then be used to define specific in vivo processes normally dependent upon JAM2.

JAM2 is expressed to a low level in many tissues and it is likely that JAM2 can be upregulated during pathological conditions. This expression pattern suggests that JAM2 localizes to endothelia. However, it is certainly possible that other cell types also express JAM2. If JAM2 localizes to the tight junction of epithelial cells, it is proposed that it plays a role during metastasis. Either defective JAM2 or decreased expression may not only decrease adhesion between tumor cells but also facilitate their movement through the endothelium into the vessel. JAM2 expression in the brain may indicate a role in the blood brain barrier. JAM2 expression in the aorta and heart indicates it may play a role during conditions which display inflammatory or permeability changes such as atherogenesis and reperfusion injury. Further, it is possible that JAM2 localizes to the intercalated discs of the myocyte and thus play a role in maintenance of the syncitium. Expression of JAM2 in the mammary gland has been noted and may play a role in the regulation of tight junctions during lactation. Alternatively, JAM2 expression in the mammary gland and prostate may play a role in malignancies of these tissues and metastasis. JAM2 transcripts are also found within the testis and may participate in the blood-testis barrier.

VI. Antibodies

The polypeptides of the present invention, fragments thereof, or cells expressing said polypeptides can be used as an immunogen to produce antibodies. These antibodies can be, for example, polyclonal or monoclonal antibodies. The present invention also includes chimeric, single chain, and humanized antibodies, as well as Fab fragments, or the product of a Fab expression library.

Antibodies generated against the polypeptides of the present invention can be obtained by administering the polypeptides to an animal, preferably a nonhuman. Even a sequence encoding only a fragment of a polypeptide of the present invention can be used to generate antibodies binding to the whole native polypeptide. Such antibodies can then be used to isolate the polypeptide from tissue expressing that polypeptide.

For preparation of monoclonal antibodies, any technique, which provides antibodies produced by continuous cell line cultures, can be used. Examples include the hybridoma technique (described by Kohler and Milstein, 1975, Nature, 256:495-497, herein incorporated by reference), the trioma technique, the human B-cell hybridoma technique (described by Kozbor et al., 1983, Immunology Today 4:72, herein incorporated by reference), and the EBV-hybridoma technique to produce human monoclonal antibodies (described by Cole, et al., 1985, in Monoclonal Antibodies and Cancer Therapy, Alan R. Liss, Inc., pp-77-96, herein incorporated by reference).

Techniques for the production of single chain antibodies, such as those described in U.S. Pat. No. 4,946,778, herein incorporated by reference, can be adapted to produce single chain antibodies to immunogenic polypeptides of the present invention.

The antibodies of the present invention can be used to:

a) Probe cellular localization/expression of JAM2 in tissues under normal and disease states.

b) Immunoprecipitate JAM2 protein from cells under control and stimulated conditions, and from tissues, control and disease, to determine whether it is modified by e.g. glycosylation, phosphorylation etc.

c) For helping determine JAM2 function. For example, if it is found that JAM2 interacts with inflammatory cells or influences their paracellular migration of inflammatory cells, neutralizing antibodies will be developed to inhibit this function both in vitro and in vivo.

d) Inhibit JAM2 interactions with JAM3 in vitro and in vivo.

e) Inhibit JAM2 interactions with α4β1/α4β7 integrins in vitro and in vivo.

VII. JAM2 Interaction with the α4β1 Integrin

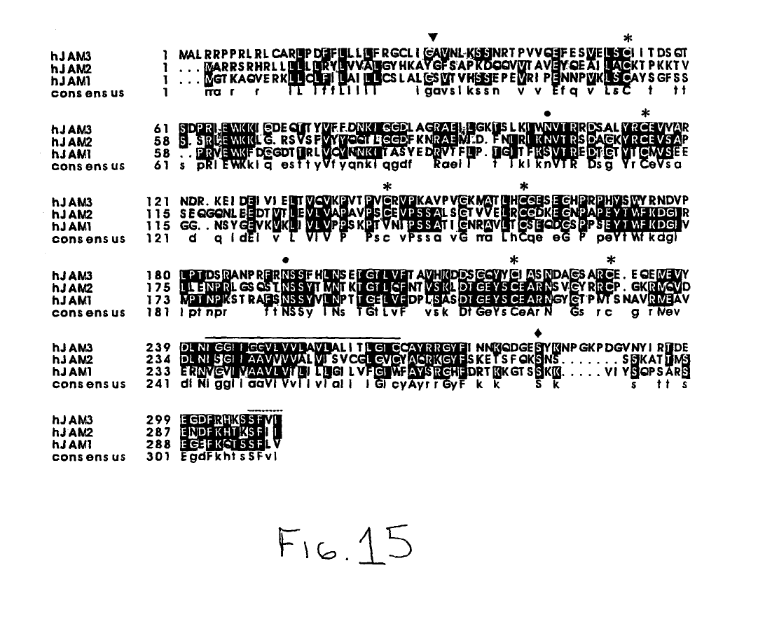

As demonstrated in the Examples, JAM2 exhibits strong heterotypic binding to a 43 kDa protein, identified and sequenced by the inventors of the present invention ((Arrate M P, Rodriguez J M, Tran T M, Brock T A, Cunningham S A, (2001) J. Biol. Chem. 276:45826-45832). This 43 kDa protein is known as human “JAM3” and was the first ligand identified for JAM2. Human JAM3 is 310 amino acids in length and is shown in FIG. 15.

JAM2 also exhibits heterotypic binding to the α4β1 integrin. More specifically, the inventors have discovered that when JAM2 binds to cell surface JAM3, JAM2 also becomes available to bind to α4β1 integrin. Therefore, JAM3 may act as a co-factor for JAM2 binding. The binding of JAM2 to α4β1 integrin does not appear to occur unless JAM2 has bound to JAM3 on the same cell membrane. As used herein, the term “α4β1 integrin” means an integrin heterodimer where the α4 subunit non-covalently associates with a P1 subunit. It is believed that JAM2 may also exhibit similar binding to the α4β7 integrin.

While not wishing to be bound by any theory, the inventors believe that the binding between JAM2 and α4β1 integrin on the cell membrane may be the result of: (a) prior adhesion of JAM3 expressed on the cell surface, with immobilized JAM2, allowing enhancement of lower affinity interaction between JAM2 and α4β1 by allowing α4β1, closer more frequent contacts with JAM2; (b) a conformation change that occurs on JAM2 after binding with JAM3 that allows its subsequent interaction with the integrin; (c) that the adhesion of JAM2 with JAM3 initiates clustering of the integrin and presentation to JAM2; (d) that JAM3 directly associates with the integrin through low affinity interactions, and that this may be regulated via JAM2/JAM3 binding; adhesion of JAM2 to JAM3 induces a conformational change of the integrin allowing its interaction with JAM2; or (e) following JAM2 adhesion to JAM3, an intracellular signaling cascade is activates that results in integrin “inside-out” signaling.

VIII. Use of Soluble JAM2 or JAM3 to Prevent JAM2-α4β1 or α4β7 Integrin Binding or Interaction

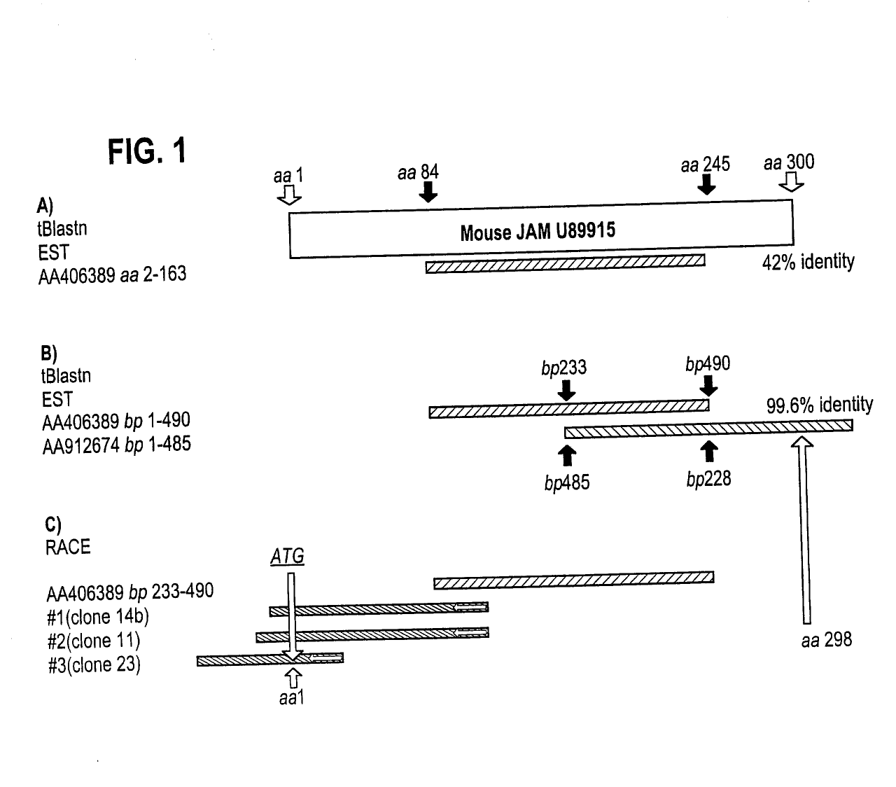

In yet another embodiment, the present invention relates to the in vivo or in vitro use of an effective amount of soluble JAM2 or soluble JAM3 to prevent JAM2-α4β1 or JAM2-α4β7 integrin binding or interaction. As used herein, the term “soluble JAM2” refers to a JAM2 polypeptide that does not contain a complete transmembrane domain (See FIG. 1). As shown in FIG. 1, the transmembrane domain of JAM2 is 23 amino acids in length. Soluble JAM2 may contain no transmembrane domain or may contain a transmembrane domain that is less than 18 amino acids in length. As used herein, the term “soluble JAM3” refers to a JAM3 polypeptide that does not contain a complete transmembrane domain (See FIG. 15). As shown in FIG. 15, the transmembrane domain of JAM3 is 23 amino acids in length. Soluble JAM3 may contain no transmembrane domain or may contain a transmembrane domain that is less than the 23 amino acids in length.

Soluble JAM2 or soluble JAM3 can be obtained using techniques known in the art. Specifically, soluble JAM2 or soluble JAM3 can be isolated and purified in its native form, using techniques known in the art. Alternatively, soluble JAM2 or soluble JAM3 can be chemically synthesized using methods known in the art, preferably solid state methods, such as the methods of Merrifield (J. Am. Chem. Soc., 85:2149-2154 (1963)). Additionally, soluble JAM2 or soluble JAM3 can be produced using methods of DNA recombinant technology (Sambrook et al., in “Molecular Cloning—A Laboratory Manual”, 2^{ND}. Ed., Cold Spring Harbor Laboratory (1989)).

An effective amount of soluble JAM2 or soluble JAM3 can be administered either in vivo (such as to a mammal (such as a human patient) in need of treatment) or in vitro (such as in an assay), to adhere to cell surface expressed JAM2 or JAM3. Possible interactions that may occur, include JAM2 or JAM3 binding to cell surface expressed JAM2, JAM3 or JAM2 binding cell surface expressed JAM3. Heterotypic adhesion of soluble JAMs with cell surface JAM2 or JAM3 will prevent subsequent heterotypic interaction of JAMs between cells. Similarly, homotypic adhesion of soluble JAMs with cell surface expressed JAM2 or JA3, may result in modulation of homotypic interaction of JAMs between cells. When the JAM2-JAM3 interaction does not occur, JAM2 binding to α4β1 or α4β7 is not apparent. As used herein, the term “cell surface bound JAM3”, refers to the expression of JAM3 in any cell type that is adhering to a JAM2 expressing cell. The cells expressing endogenous JAM2 and JAM3 may be of the same type i.e. both may be endothelial cells. Alternatively, the cell types may be different. For example, one may be an endothelial cell and one may be a leukocyte. Moreover, the adhesion of JAM2 with JAM3 laterally, within the same cell may occur. Soluble JAM2 or soluble JAM3 can also be administered to prevent this interaction as well. An effective amount of soluble JAM2 or soluble JAM3 that is administered to prevent the hereinbefore described JAM2-α4β1/α4β7 integrin binding can be determined by those of ordinary skill using routine techniques known in the art.

IX. Screening Assays for Identifying Compounds that Modulate JAM2-α4β1/α4β7 Integrin Binding

In yet another embodiment, the present invention relates to in vitro screening assays for identifying compounds that disrupt or modulate JAM2-α4β1/α4β7 integrin binding. The assays of the present invention can be conducted using techniques known in the art, and preferably are amenable to high-throughput screening of chemical libraries. Suitable candidate compounds that can be used in the methods of the present invention include any molecule, such as, but not limited to, proteins, oligopeptides, small organic molecules, polysaccharides, oligonucleotides (sense or antisense), polynucleotide (sense or antisense), etc. The candidate compound can encompass numerous chemical classes, though they are typically organic molecules. The candidate compound can be obtained for a wide variety of sources including libraries of synthetic or natural compounds. For example, many methods are known in the art for the random and directed synthesis of a wide variety of organic compounds and biomolecules, including expression of randomized oligonucleotides. Alternatively, libraries of natural compounds in the form of bacterial, fungal, plant and animal extracts can be used. Additionally, natural or synthetically produced libraries and compounds are readily modified through conventional chemical, physical and biochemical methods.

The assays of the present invention involve preparing a reaction mixture containing either an α4β1/α4β7 integrin or JAM2. Optionally, the reaction mixture can also contain JAM3. The reaction mixture is contacted with its binding partner (i.e., if the reaction mixture contains JAM2, then the reaction mixture can be contacted with an α4β1/α4β7 integrin. If the reaction mixture contains an α4β1/α4β7 integrin, then the reaction mixture can be contacted with JAM2) in the presence and absence of the candidate compound under conditions for a time to allow the components of the reaction mixture to interact and bind, thus forming a complex which can be removed and/or detected in the reaction mixture. The candidate compound may be initially included in the reaction mixture or may be added subsequent to the addition of the JAM2 and α4β1/α4β7 integrin. A control reaction mixture is incubated without the candidate compound or with a placebo. The formation of any complexes between JAM2 and the α4β1/α4β7 integrin is detected. The formation of a complex in the control reaction, but not in the reaction mixture containing the candidate compound, indicates that the compound interferes with the binding interaction between JAM2 and the α4β1/α4β7 integrin. Additionally, complex formation within reaction mixtures containing the candidate compound and JAM2 or α4β1/α4β7 integrin can also be compared to complex formation within reaction mixtures containing the candidate compound and a mutant JAM2 or α4β1/α4β7 integrin. This comparison may be useful in identifying compounds that disrupt interactions of mutants but not wildtype JAM2 or α4β1/α4β7 integrin. After the reaction is completed, the unreacted components are removed (such as by washing) under conditions such that any complexes formed will remain immobilized on the solid surface. In view of decreased or increased binding between the α4β1/α4β7 integrin and JAM2 in the presence or absence of the candidate compound a determination is made whether or not the candidate compound modulates the binding between the JAM2 and the α4β1/α4β7 integrin.

The assays of the present invention can be conducted in a heterogeneous or homogeneous format. Heterogeneous assays involve anchoring either the JAM2 or α4 β1/α4β7 integrin onto a solid phase and then detecting complexes anchored on the solid phase at the end of the reaction. In homogeneous assays, the entire reaction is carried out in a liquid phase. With either approach, the order of addition of reactants can be varied to obtain different information about the candidate compounds being tested. For example, candidate compounds that interfere with the interaction between JAM2 and α4β1/α4β7 integrins, by competition, can be identified by conducting the reaction in the presence of the candidate compound, namely, by adding the candidate compound to the reaction mixture prior to or simultaneously with JAM2 and α4β1/α4β7 integrin. Alternatively, candidate compounds can be added to the reaction mixture after complexes have been formed. Moreover, with either approach, JAM3 can be added to the reaction to improve the specificity and/or sensitivity of the reaction.

For example, in a heterogeneous assay, JAM2 or α4β1/α4β7 integrin is immobilized (i.e. anchored) on a solid phase by covalent or non-covalent attachments (the immobilized binding partner). Examples of a solid phase that can be used include, but not limited to, glass, polystyrene, polypropylene, polyethylene, dextran, nylon, amylases, natural and modified celluloses, polyacrylamides, gabbros, and magnetite. The solid phase may be spherical, as in a bead, or cylindrical, as in the inside surface of a test tube, or the external surface of a rod. Alternatively, the surface may be flat such a sheet, test strip, etc. Preferred solid phases include microtiter plates and sepharose beads. Those skilled in the art will be able to ascertain the appropriate solid phase using routine techniques known in the art.

The non-immobilized component (or non-immobilized binding partner) is labeled either directly or indirectly. Examples of labels that can be used include, but are not limited to, enzyme labels, such as glucose oxidase, alkaline phosphatase, radioisotopes, such as iodine, carbon, sulfur, tritium, indium and technetium and fluorescent labels such as fluorescein and rhodamine and green fluorescent protein (GFP) and its variants, or Cy dyes such as Cy3 and Cy5 and biotin. The immobilized binding partner (either the JAM2 or α4β1/α4β7 integrin) is then contacted with the labeled binding partner in the presence and absence of candidate compound that is believed to be capable of specifically reacting with JAM2 or α4β1/α4β7 integrin. After the reaction is complete, unreacted components are removed (such as by washing with buffers) and any complexes formed remain immobilized on the solid phase. The detection of the complex anchored on the solid surface can be accomplished in a number of ways. Where the non-immobilized binding partner is pre-labeled, the detection of the label immobilized on the surface indicates that complexes were formed. Where the non-immobilized binding partner was not pre-labeled, an indirect label can be used to detect complexes anchored on the solid phase, for example, by using a labeled antibody specific for the non-immobilized binding partner (the antibody, may be directly labeled or indirectly labeled with a labeled anti-Ig antibody). Depending upon the order of addition of reaction components, candidate compounds that inhibit complex formation or which disrupt preformed complexes can be detected.

Alternatively, the reaction can be conducted in a liquid phase in the presence or absence of the candidate compound, the reaction products separated from unreacted components, and complexes detected. For example, the complexes can be detected using an immobilized antibody specific for one of the binding components to anchor any complexes formed in solution, and a labeled antibody specific for the other partner to detect immobilized complexes. Depending upon the order of addition of reactants to the liquid phase, test compounds which inhibit complex or which disrupt preformed complexes can be identified.

For example, in a homogeneous assay, a preformed complex of JAM2 and α4β1/α4β7 integrin are prepared in which either the JAM2 or α4β1/α4β7 integrin is labeled, but the signal generated by the label is quenched due to complex formation. The addition of a candidate compound that competes with and displaces one of the species from the preformed complex will result in the generation of a signal above background. Therefore, candidate compounds that disrupt the JAM2/α4β1 or α4β7 integrin binding interaction can be identified.

Using other methodologies, in a homogeneous assay, JAM2 and α4β1 or α4β7 integrin can be conjugated, either directly or indirectly to donor and acceptor fluorophore beads respectively, such as those composing the AlphaScreen technology. Upon adhesion of JAM2 with JAM3, the beads will be brought into proximity. A laser is employed to excite for example the donor bead conjugated to JAM2, which results in conversion of ambient oxygen to a more excited singlet state. The singlet state oxygen molecules diffuse across and react with a chemiluminescer in the acceptor bead conjugated to JAM3. Further activation of fluorophores contained within the same bead, emit light at 520-620 nm. Alternatively, other donor or acceptor pairs can be used.

Using other methodologies, JAM2 and α4β1/α4β7 integrin may be fused with each of EGFP (enhanced green fluorescent protein) and EBFP (enhanced blue fluorescent protein). EBFP and EGFP mutants of GFP, when in close proximity to one another and can act as a fluorescence resonance energy transfer (FRET) pair. Thus, upon mixing of JAM2 and α4β1/α4β7 integrin complexes will form. Excitation of the JAM-EBFP will result in emissions that excite the JAM-EGFP, and thus the extent of complex formation can be monitored in the presence or absence of compound.

The JAM2 and/or α4β/α4β7 integrin and optionally, JAM3 used in the assays of the present invention can be prepared using recombinant DNA techniques described herein. For example, recombinant cells expressing JAM2, and/or α4β1/α4β7 integrin and optionally JAM3 can be used. Alternatively, JAM2 or JAM3 can be isolated and purified in its native form or chemically synthesized using methods known in the art. Additionally, JAM2 and/or α4β1/α4β7 integrin fusion proteins and fragments of JAM2 and/or α4β1/α4β7 integrin that correspond to the binding domains of JAM2 and/or α4β1/α4β7 integrin can also be used in the assays described herein. Alternatively, the JAM2 and/or α4β1/α4β7 integrin and optionally, JAM3 used in the assay described herein can be expressed on the surface of a cell.

X. Pharmaceutical Compositions

In another embodiment, the present invention relates to certain compounds and pharmaceutical compositions that can be administered to a mammal in need of treatment. One type of compound that can be administered to a mammal alone or in a pharmaceutical composition, where it is mixed with suitable carriers or excipients, is soluble JAM2 or soluble JAM3. Soluble JAM2 or soluble JAM3 modulates or interferes with JAM2-α4β1/α4β7 integrin binding. Soluble JAM2 or soluble JAM3 can be isolated and purified in its native form, using techniques known in the art. Alternatively, soluble JAM2 or soluble JAM3 can be chemically synthesized using methods known in the art, preferably solid state methods, such as the methods of Merrifield (J. Am. Chem. Soc., 85:2149-2154 (1963)). Additionally, soluble JAM2 or soluble JAM3 can be produced using methods of DNA recombinant technology (Sambrook et al., in “Molecular Cloning—A Laboratory Manual”, 2^{ND}. Ed., Cold Spring Harbor Laboratory (1989)). Additionally, soluble JAM2 or soluble JAM3 fusion proteins or fragments of soluble JAM2 or soluble JAM3 can also be used. For example, peptides about 5 to about 25 amino acids in length, linear or cyclized, derived from the sequences of JAM2 and JAM3, that are found to prevent either the adhesion of JAM2 with α4β1, can be used. These fusion proteins or fragments of JAM2 or JAM3 can be made using the techniques described herein. Other compounds that can be administered to a mammal alone or in a pharmaceutical composition are the candidate compounds identified pursuant to the assays described herein.

Suitable excipients that can be used in the pharmaceutical composition of the present invention, include but are not limited to, fillers such as sugars, including lactose, sucrose, mannitol, sorbitol, and the like, cellulose preparations such as, maize starch, wheat starch, rice starch, potato starch, gelatin, gum tragacanth, ethyl cellulose, hydroxypropylmethylcellulose, sodium carboxymethylcellulose, polyvinylpyrrolidone (PVP), and the like, as well as mixtures of any two or more. Optionally, disintegrating agents can be included, such as cross-linked polyvinyl pyrrolidone, agar, alginic acid or a salt thereof, such as sodium alginate and the like.

In addition to the excipients, the pharmaceutical composition can include one or more of the following, carrier proteins such as serum albumin, buffers, binding agents, sweeteners and other flavoring agents; coloring agents and polyethylene glycol.

Suitable routes of administration for the compound or pharmaceutical composition include, but are not limited to, oral, rectal, transdermal, vaginal, transmucosal or intestinal administration, parenteral delivery, including intramuscular, subcutaneous, intramedullary injections, as well as intrathecal, direct intraventricular, intravenous, intraperitoneal, intranasal, intraocular, and the like.

For oral administration, the pharmaceutical composition can be formulated as tablets, pills, capsules, dragees, liquids, gels, syrups, slurries, suspensions and the like. For administration by injection, the compound or the pharmaceutical composition can be formulated in an aqueous solution. Preferably, the aqueous solution is in a physiologically compatible buffer such as Hank's solution, ringer's solution or a physiological saline buffer.

The pharmaceutical composition of the present invention can be manufactured using techniques known in the art, such as, but not limited to, conventional mixing, dissolving, granulating, dragee-making, levigating, emulsifying, encapsulating, entrapping, lyosphilizing processes or the like.

XI. Methods of Treatment

The present invention also relates to methods of treating a mammal with the previously described compounds or pharmaceutical compositions for the purpose of preventing the JAM2-α4β1/α4β7 integrin mediated interaction. It is believed preventing such interaction can be used to treat diseases associated with leukocyte adhesion to the endothelium and their subsequent diapedesis through the vessel wall. This phenomenon commonly occurs during inflammation and accompanies autoimmune diseases. Therefore, the compounds identified pursuant to the assays of the present invention can be used to treat conditions or disorders, including, but not limited to, systemic lupus erythematosis, rheumatoid arthritis, juvenile chronic arthritis, spondyloarthropathies, systemic sclerosis (scleroderma), idiopathic inflammatory myopathies (including, dermatomyositis, polymyositis, etc), Sjsgren's syndrome, systemic vasculitis, sarcoidosis, autoimmune hemolytic anemia (including, immune pancytopenia, paroxysmal nocturnal hemoglobinuria), autoimmune thrombocytopenia (including idiopathic thrombocytopenic purpum, immune-mediated thrombocytopenia), thryoiditis (including Grave's disease, Hashimoto's thyroiditis, juvenile lymphocytic thyroiditis, atrophic thyroiditis), diabetes mellitus, immune-mediated renal disease (including glomerulonephritis, tubulinterstitial nephritis), demyelinating diseases of the central and peripheral nervous system, including multiple sclerosis, idiopathic demyelinating polyneuropathy or Guillain-Barre syndrome, chronic inflammatory demyelinating polyneuropathy), hepatobiliary diseases (including hepatitis A-E and other non-hepatotropic viruses), autoimmune chronic active hepatitis, primary biliary cirrhosis, granulomatous hepatitis, and sclerosing cholangitis, inflammatory and fibrotic lung diseases (including inflammatory bowel disease, ulcerative colitis, Crohn's disease), gluten-sensitive enteropathy, Whipple's disease, autoimmune or immune-mediated skin diseases (including bullous skin diseases, erythema multiforme and contact dermatitis, psoriasis), allergic disease (including asthma, allergic rhinitis, atopic dermatitis, food hypersensitivity and urticaria), immunologic disease of the lung (including eosinophilic pneumonias, idiopathic pulmonary fibrosis, hypersensitivity, pneumonitis, transplantation associated diseases (including graft rejection and graft-versus-host-disease).

The treatment method of the present invention involves administering to a mammal the compound or pharmaceutical composition in a therapeutically effective amount sufficient to treat the conditions or disease in question. As used herein, the term “therapeutically effective amount” means an amount that produces the effects for which it is administered. The exact dose will be ascertainable by one skilled in the art. As known in the art, adjustments based on age, body weight, sex, diet, time of administration, drug interaction and severity of condition may be necessary and will be ascertainable with routine experimentation by those skilled in the art.

Suitable routes of administration for the compound or pharmaceutical composition include, but are not limited to, oral, rectal, transdermal, vaginal, transmucosal or intestinal administration, parenteral delivery, including intramuscular, subcutaneous, intramedullary injections, as well as intrathecal, direct intraventricular, intravenous, intraperitoneal, intranasal, intraocular, and the like.

By way of example, and not of limitation, examples of the present invention shall now be given.

### EXAMPLE 1

### Cloning and Expression of Human JAM2

The polynucleotide sequence shown in SEQ ID NO:1 was cloned using a combination of electronic and conventional cloning techniques. The electronic techniques used involved utilizing the Expressed Sequence Tag (EST) databases accessed through the home page of the National Center for Biotechnology Information (NCBI) at www.ncbi.nlm.nih.gov. As a template for electronic cloning, the cDNA sequence of a novel mouse Junctional Adhesion Protein (JAM) published by Martin-Padura I, Lostaglio S, Schneemann M, Williams L, Romano M, Fruscella P, Panzeri C, Stoppacciaro A, Ruco L, Villa A, Simmons D, Dejana E, J. Cell. Biol. (1998) 142(1): 117-27, herein incorporated by reference, was used. The mouse JAM cDNA sequence is also available on GenBank (Accession #U89915). The advanced Basic Local Alignment Search Tool (BLAST 2.0) was used to identify ESTs displaying homology with mouse JAM.

Electronic Cloning

The complete mouse JAM peptide sequence (GenBank Accession # U89915) was searched for homology with human EST sequences using the tblastn program which compares a protein query sequence against a nucleotide sequence database dynamically translated in all reading frames. The complete mouse JAM protein sequence is 300 amino acids in length. The initiation codon begins at base pair (herein after “bp”) 71 and the stop codon at 971 (see FIG. 1A). Of the EST hits, one was chosen for further analysis. The criteria used were reasonable homology to mouse JAM and conservation of the cysteine residues specific to the immunoglobulin-like fold. AA406389 showed 42% identity at the amino acid level with mouse JAM over a 161 amino acid overlap and was chosen for assembly of a virtual cDNA (see FIG. 1A). Throughout assembly, translation was monitored in all reading frames to identify the putative codons for initiation and termination of the virtual protein. Where possible, examination of multiple overlapping ESTs was conducted in order to identify sequencing errors. The final 3′ 130 by of AA406389 was blasted through the human dbEST using the blastn program. AA912674 showed 99.6% identity over a 257 bp overlap (see FIG. 1B). Further searching in the database for sequence at the 5′-end of AA406389 did not reveal additional ESTs.

Conventional Cloning

In order to obtain further 5′ sequence for this cDNA, RACE was performed. The prime purpose was to identify a putative ribosome start site (ATG) that coincided approximately in a linear sequence with that in mouse JAM.

Three separate RACE reactions were performed consecutively using the Marathon cDNA amplification kit (Clontech, Palo Alto, Calif.) on human placental mRNA (Clontech). The first was performed with an oligonucleotide, 5′-CCCCGCATCACTTCTTGTCACATTTTTGATCCGG-3′ (SEQ ID NO:3), directed against AA406389. An alignment of this primer with mouse JAM positioned it some 318 bp downstream of the translational start site. mRNA was reverse transcribed with AMV reverse transcriptase (Clontech) at 42° C. RACE was performed according to the following protocol:

| Cycles # | Temperature° C. | Time |
|---|---|---|
| 1 | 94 | 30 sec |
| 5 | 94 | 5 s |
| | 72 | 4 min |
| 5 | 94 | 5 s |
| | 70 | 4 min |
| 25 | 94 | 5 s |
| | 68 | 4 min |

Products were ligated into the E. coli vector pCRII-TOPO (Invitrogen, Carlsbad, Calif.) and eleven clones selected for sequencing (ABI sequencer, Seqwright, Tex.). The longest clone extended 45 bps 5′ of an ATG that approximately aligned with that of mouse JAM (see FIG. 1C). However, a STOP codon upstream of this putative translational initiation codon could not be identified.

In an attempt to identify a STOP codon, in frame and upstream of this ATG, two additional RACE reactions were performed. The first used the same primer for extension as RACE reaction 1. However, mRNA was reverse transcribed with thermoscript (BRL Life Technologies, New York, U.S.A.) at 58° C. Products were ligated into pCR-Blunt II—TOPO (Invitrogen). Of six clones sequenced, the longest only possessed 22 additional base pairs (FIG. 1C). For RACE reaction 3, an oligonucleotide was designed within the sequence obtained from RACE reaction 2,5′-CTGCTCTGAGGAGGTCGAGGGTCCC-3′ (SEQ ID NO:4). The mRNA was transcribed with thermoscript at 58° C. Three clones were sequenced and the longest possessed 167 additional base pairs to that identified in RACE reaction 2 (see FIG. 1C).

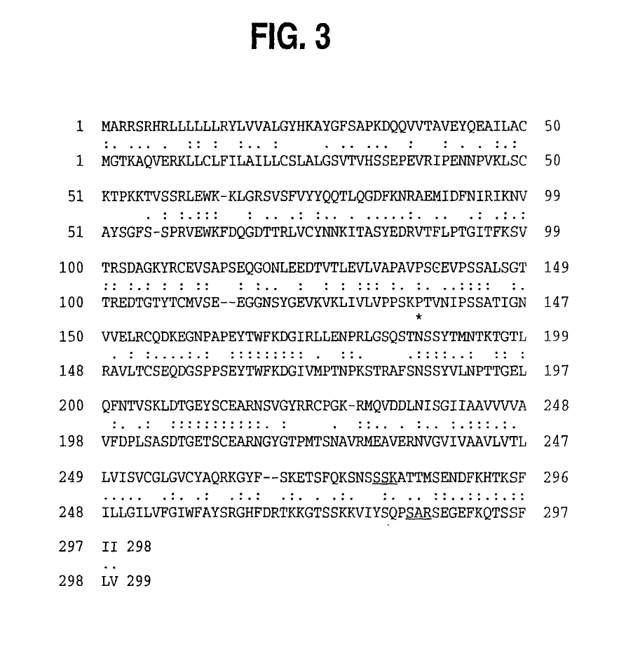

RACE reactions produced in total an additional 234 by 5′ of the putative translational initiation codon. A stop codon was not identified within this sequence that was in frame with the ATG. However, the inventors believe it to be the true start of the open reading frame for several reasons. First, alignment of the human JAM2 reading frame with the published mouse JAM reading frame (see FIG. 3) shows that this ATG approximately coincides with that of mouse JAM. Second, the nucleotides surrounding this site (GGAAGATGG) possess an A at the −3 position and a G at the +4 position thus conforming to the initiation consensus sequence. Third, the first 28 amino acids of JAM2 predict a signal peptide.

Construction of a Full-Length JAM2

In order to construct full-length human JAM2, the products of two separate PCR reactions were ligated together via an internal EcoNI restriction site. For the synthesis of the 5′-section of the open reading frame, a sense primer encompassing the initiation codon, 5′-GCCGCGGATCCAAGATGGCGAGGAGG-3′ (SEQ ID NO:5) and an antisense primer targeted at the end of the extracellular domain, 5′-GCTATTATGCCGGTACCGTTGAGATCATCTAC-3′ (SEQ ID NO:6), were designed (restriction sites incorporated into the primers for subsequent manipulation are underlined). A product was amplified from human placental mRNA (Clontech) using the following program: 2 min at 95° C., 1 cycle; 20 s at 95° C., 20 s at 58° C., 30 s at 72° C., 35 cycles; 3 min at 72° C., 1 cycle. The approximate 720 by product was ligated into pCR II—TOPO.

For synthesis of the 3′-section of the open reading frame sense primer, 5′-TAAAAATCGAGCTGAGATGATAG-3′ (SEQ ID NO:7), located 248 bp into the reading frame was coupled with antisense primer, 5′-TTAAATTATAAAGGATTTTGTG-3′ (SEQ ID NO:8), that incorporated the stop codon (bold). A product was amplified from mRNA derived from human embryonic kidney cells (HEK-293, available from the American Type Culture Collection, Manassus, Va., ATCC Accession #CRL-1573) using the following program: 7 min at 95° C., 1 cycle; 20 s at 95° C., 20 s at 56° C., 30 s at 72° C., 28 cycles; 5 min at 72° C., 1 cycle. The approximate 649 by product was ligated into pCR II—TOPO. Two independent PCR reactions were performed and a clone from each sequenced for verification of each base.

Sequence Features

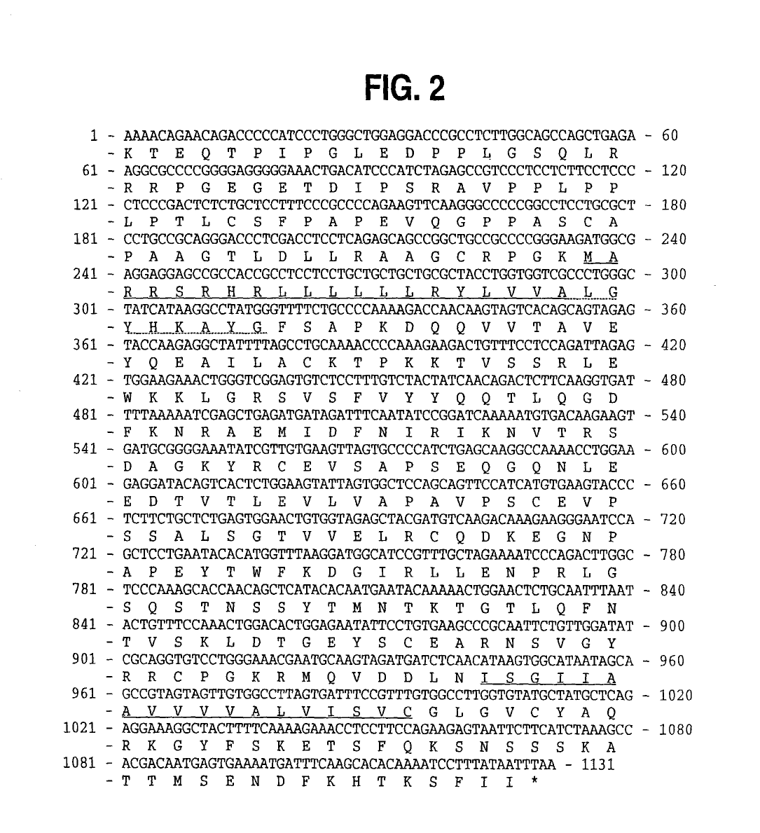

The human JAM2 nucleotide and amino acid sequence is shown in FIG. 2 and in SEQ NOS: 1 and 2, respectively. As shown in FIG. 2, the complete coding region of 298 amino acids features a putative signal sequence, two immunoglobulin-like domains, a single transmembrane domain (underlined) and a short intracellular domain. There are two possible cleavage sites for the signal peptide i.e. VVA-LG (single underline) or AYG-FS (dotted underline). The designated ATG is the true translational initiation signal based on the fact that it lies within a Kozak consensus and it aligns with human JAM1 ATG. The four cysteine residues predicted to form disulfide bonds within the immunoglobulin-like domains are highlighted. The 1^{st} and 2^{nd} cysteine are located in the first immunoglobulin-like fold and the 3^{rd} and 4^{th} in the second immunoglobulin-like fold. Highlighted are potential N-linked glycosylation sites (NxS/T) at amino acids #98, #187, #236 and a potential PKC phosphorylation site (S/TxR/K) at amino acid #279. Thus, JAM2 function may be modified by PKC. Further, the amino acids at the extreme C-terminus of JAM2 (SFII) conform to a consensus that would be predicted to interact with PDZ domains (Songyang Z, Fanning A S, Fu C, Xu J, Marfatia S M, Chishti A H, Crompton A, Chan A C, Anderson, J M, Cantley, L C (1997) Science 275:73-77). Proteins containing PDZ domains are predominantly localized to the plasma membrane and are recruited to specialized sites of cell-cell contact. Most recently, it has been reported that the intracellular domain of human JAM (JAM1) binds to the tight junction associated proteins ZO-1 and AF-6 via their PDZ domains (Bazzoni G, Martinez-Estrada O M, Orsenigo F, Cordenonsi M, Citi S, Dejana E, (2000) J. Biol. Chem. 275:20520-20526; Ebnet K, Schulz C U, Meyer Zu, Brickwedde M K, Pendl G G, Vestweber D. (2000) J. Biol Chem June 15; [epub ahead of print]). Thus it is highly likely that JAM2 will display similar binding activities.

Sequence Alignment

An alignment of the human junctional adhesion sequence with mouse JAM reveals 43% similarity and 35% identity at the amino acid level (see FIG. 3). The positions of the conserved cysteines are highlighted in both sequences.

Expression Pattern

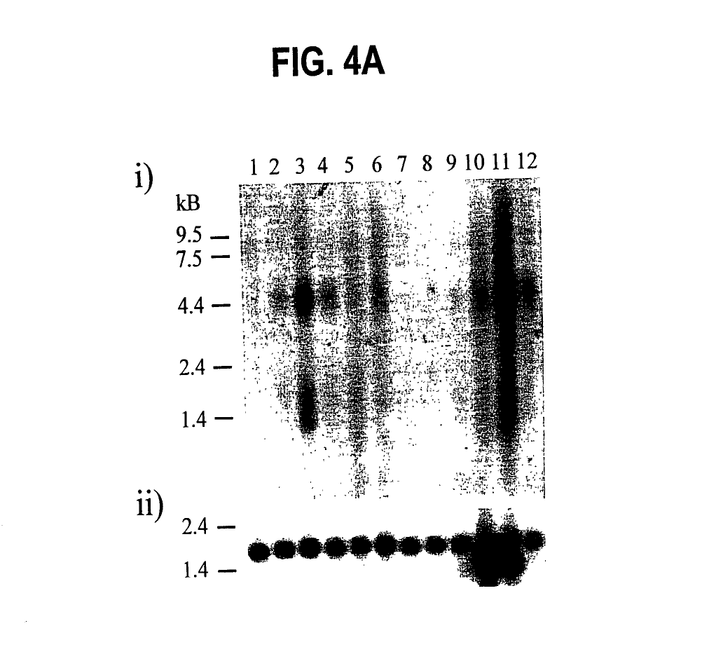

Tissue expression of JAM2 was examined on a normalized human Multiple Tissue Northern blot (Clontech) with an [α³²P]dCTP labeled probe derived from the extracellular domain. The results show that JAM2 is expressed as two transcripts of approximately 4.5 kb and 1.5 kb (see FIG. 4). The blots were probed at high stringency and thus these two species likely represent alternatively spliced products. FIG. 4 shows that human JAM2 is abundantly expressed in the heart. Expression also occurs in the placenta with much lower levels apparent in brain and skeletal muscle. FIG. 4B shows a more detailed examination of the JAM2 transcript in the heart. A clear chamber specific expression was not apparent. Relative to GAPDH, there is somewhat lower expression in fetal heart. However, major differences in the aorta, atrium and ventricles were not observed.

TABLE 1
Expression Characteristics of Human JAM2

| mRNA Source | EST, GenBank Acc # | RT-PCR |
|---|---|---|
| Tissue | | |
| Mix of melanocyte, fetal heart, | AA406389, AA410345 | |
| pregnant uterus | | |
| Mix of fetal liver & spleen | AI052637 | |
| Mix of fetal lung, testis, B cell | AA912674, AI017553 | |
| Embryo (total) | W80145 | |
| Brain, anaplastic oligodendroma | AI199779 | |
| Lung, fetal/adult | N90730, T89217 | |
| Kidney, normal/tumor | AA865038, AA987434 | |
| Prostate | AI201753 | |
| Heart fetal/4 weeks | AI140139, AA445150 | |
| Mammary (4 weeks) | AI154320, AI690843 | |
| Testis | AA725566 | |
| Placenta | | + |
| Endothelial Cells | | |
| Human Umbilical Vein | | − |
| Human Umbilical Vein, | | + |
| immortal (ECV) | | |
| Human Aortic | | + |
| Human Cardiac Microvascular | | + |
| Epithelial Cells | | |
| Human embryonic kidney | | + |
| (HEK-293) | | |
| Colonic adenocarcinoma, | | v. low |
| CaCo-2 | | |

While not wishing to be bound by any theory, due to the homology of JAM2 with mouse JAM, the inventors predict that JAM2 localizes to the endothelial cells of these tissues. This is confirmed by PCR analysis of mRNA derived from human aortic endothelial cells and cardiac microvascular endothelial cells (see Table 1, above). Interestingly, a product from human umbilical vein endothelial cells (hereinafter referred to as “HUVEC”) was barely detectable. Thus, JAM2 expression may be restricted to certain vascular beds. In addition to the endothelium, mouse JAM is also expressed in select epithelial cells. Using the polymerase chain reaction, expression in human embryonic kidney cell line (HEK-293) can be detected but only very low levels in the colonic epithelial cell line, CaCo2.

The EST database contains many ESTs that partially encode the human JAM2 sequence. Table 1 documents the tissues from which sequence was derived. It does not provide information about the level of expression in each tissue.

### EXAMPLE 2

### Functional Properties of JAM2

A. Methods

1. Expression of Extracellular Domain in Insect Cells

Oligonucleotides were designed to amplify the extracellular domain of human JAM2 from the full-length clone. Sense 5′-GCCGCGGATCCAAGATGGCGAGGAGG-3′(SEQ ID NO:5) and antisense 5′-GCTATTATGCCGGTACCGTTGAGATCATC-3′(SEQ ID NO:6) oligonucleotides incorporated BamHI and KpnI restriction sites (underlined) for subcloning of the product into a pFastBac1 (Life Technologies, GIBCO BRL, Grand Island, N.Y.) vector that possessed the constant region of mouse IgG-2a (Cunningham S A, Tran T M, Arrate M P, Brock T A, (1999) J. Biol. Chem. 274:18421-7). This vector drives protein expression from the polyhedrin promotor. The recombinant protein is secreted from the Sf21 insect cells as a fusion to mIgG2a.

2. Expression of the Full Length Clone in Mammalian Cells

The full-length clone of JAM2 was modified at its C-terminus by PCR mutagenesis to incorporate an HA-Tag for detection purposes. The sense 5′-GCCGCGGATCCAAGATGGCGAGGAGG-3′ (SEQ ID NO:5) oligonucleotide contained a BamHI site (underlined) for subsequent manipulation. The antisense 5′-TCAGGCGTAGTCGGGCACGTCGTAGGGGTAAATTATAAAGGATTTTGTGTGC-3′(SEQ ID NO:9) oligonucleotide incorporated a stop codon (underlined) and sequence (italics) that specified the HA-tag amino acids, YPYDVPDYA, (SEQ ID NO: 10) to be inserted. JAM2-HA, modified in the pGEM-7 (Promega, Madison, Wis.) vector, was digested with BamHI and XhoI (polylinker) and ligated into the BamHI and XhoI sites of pcDNA6/VS-His (B) (Invitrogen, Carlsbad, Calif.). This vector utilizes the CMV promoter to drive protein expression.

CHO-K 1 cells were transfected with either 10 μg of vector possessing no insert, or pcDNA6-JAM2 using FuGENE™ 6 reagent (Roche Diagnostics Corporation, Indianapolis, Ind.). Stable cells lines, control and JAM2, were selected with 5-10 μg/ml of Blasticidin. For Western blot analysis, cells were lysed in 1% TX-100 buffer in the presence of protease inhibitors (cocktail set III, Calbiochem, La Jolla, Calif.). Some 36 μg of protein was electrophoresed through 10% polyacrylamide gels and probed with 1:2000×dilution of preimmune or anti-JAM2 polyclonal serum. Specific bands were viewed using enhanced chemiluminesence with 1:30,000×dilution of GAM-HRP (Fischer, Pittsburgh, Pa.).

3. Chromosomal Localization and Intron/Exon Boundaries

In order to identify genomic sequence, the public non-redundant database was searched using the Blastn program with JAM2 cDNA sequence. The results required minor manual modification due to dual designation of isolated bases at the end of some exon boundaries. The correct designation was based on 5′ and 3′ splice-site consensus sequences. It was possible to confirm all intron/exon boundaries by retrieving identical information from more than one deposit of genomic sequence.

4. Antibodies

Female BALB/c mice (8-week-old; Harlan, Indianapolis, Ind.) were immunized and then boosted 3×, 28 days apart, by intraperitoneal and subcutaneous injections of 100 μg purified JAM2 extracellular domain emulsified with an equal volume of Freund's adjuvant. Complete Freund's adjuvant was used for the first immunization and incomplete Freund's adjuvant for subsequent injections. Serum was collected 10 days following each boost.

5. Immunofluorescence

CHO-K1, control or JAM2 expressing, grown on glass slides to confluence, were fixed with 1% paraformaldehyde and stained with 1:100×dilution of either preimmune or anti-JAM2 mouse polyclonal serum. GAM-FITC at 1:100×was used as secondary. Fluorescence was viewed using a Noran™ Confocal laser-scanning microscope (Koran Instruments, Middleton, Wis.) equipped with argon laser and appropriate optics and filter module for FITC detection. Digital images were obtained at x400 using a 0.75N/A Nikon x20 lens. A Z-axis motor attached to the inverted microscope stage was calibrated to move the plane of focus at 0.4 μm steps through the sample. Collected 12-bit grey scale images at 512×480 resolution, stored on a re-writable optical hard disk, were volumetrically reconstructed using the Image-1/Metamorph™ 3-D module (Universal Imaging Corp., Brandywine Parkway, Pa.).

6. Adhesion Assay

In vitro adhesion assays were performed in 96 well plates essentially as described in Todderud, G., J. J. Leukoc. Biol. 52:85 (1992), herein incorporated by reference. Briefly, 50 μl of goat anti-mouse IgG2a was coated at 5 μg/ml in PBS and used to capture 4.8 pmoles of JAM2-Fc or mIgG2a (control). Various leukocyte cell lines i.e. T lymphocytes, HSB, HPB-ALL; B lymphocytes, RAMOS; monocytic cells, HL60, THP-1, and the erythroleukemic, K562 lines were labeled with calcein (Molecular Probes Inc., Eugene, Oreg.) at 50 μg/ml for 25 minutes at 37° C. with 250,000 cells/well in binding buffer that consisted of Tris buffered saline plus 1 mM each of CaCl₂, MgCl₂ and MnCl₂. Wells were washed 3×, lysed with 50 mM Tris (pH 7.5), 5 mM EDTA, 1% NP40, and fluorescence read in a Cytofluor with excitation at 485/20 nm and emission at 530/25 nm. Specific binding was calculated as fluorescence with JAM2-Fc minus fluorescence with mIgG2a. For antibody inhibition, protein captured on wells or HSB cells were incubated for 30 min at RT in binding buffer with 1:100×dilution of preimmune (normal mouse serum) or anti-JAM2 mouse polyclonal serum. Following incubation, excess antibody was removed by washing 3×prior to continuation of the assay. Overall differences among experimental groups for each parameter were first assessed by one-way analysis of variance (ANOVA) and individual pairwise group comparisons were analyzed by Fisher's protected least significance difference (PLSD) post hoc test.

7. Cell Surface Biotinylation

HSB or K562 cells were surface biotinylated using EZ-Link Sulfo-NHS-Biotin (Pierce, Rockford, Ill.) according to the manufacturer's instructions. Cells (2.5×10⁷/ml) were washed 3×following incubation with 0.5 mg/ml Sulfo-NHS-Biotin for 30 min at RT. Cell lysis was achieved in Tris buffered saline (pH 7.5), 1% Triton X-100, 1 mM MnCl₂, 1 mM MgCl₂, 1 mM CaCl₂ with the inclusion of Protease Inhibitor Cocktail Set III (Calbiochem, La Jolla, Calif.). Some 5 μg of JAM-Fc fusion was added to approximately 1 mg of lysis and incubated at 4° C. ON. Proteins bound to JAM were precipitated with Protein A sepharose (30 μl), boiled 5 minutes with 10 mM DTT in SDS sample buffer and separated on 9% SDS gels. Following transfer to PDVF membrane, biotinylated proteins were detected using streptavidin-HRP (1:4000) and enhanced chemiluminescence (ECL) (Amersham Pharmacia Biotech, Piscataway, N.J.).

B. Results

JAM2 was mapped to chromosome 21 at position q21.2 using the public database. Sequence was retrieved at 100% identity from two contiguous non-overlapping sequences of 100,000 bp each (Accession No. AP000087.1 and AP000086.1). The coding region of JAM2, which constitutes 897 bp, is distributed over 10 exons as shown below in Table 2.

TABLE 2

| 3′ splice | Exon No. | Exon (bp) | 5′ splice | Intron (bp) |
|---|---|---|---|---|
| nnnnnn/(N) | 1 | >305 | TGGGCT/gtaagt | 43,994 |
| tttcag/ATCATA | 2 | 66 | ACCAAG/gtacag | 5,916 |
| tcctag/AGGCTA | 3 | 108 | TTCAAG/gtaagc | 3,781 |
| taaaag/GTGATT | 4 | 153 | TATTAG/gtgatg | 4,767 |
| gttcag/TGGCTC | 5 | 203 | ACTCTG/gtaagg | 3,290 |
| aaatag/CAATTT | 6 | 100 | AAGTAG/gtaagc | 3,709 |
| ttccag/ATGATC | 7 | 108 | TTTCAA/gtaagt | 3,347 |
| ttgtag/AAGAAA | 8 | 16 | CTTCCA/gtaagt | 2,890 |
| aaacag/GAAGAG | 9 | 43 | GAAAAT/gtgagt | 2,256 |
| tcctag/GATTTC | 10 | >221 | NNNNNN/(n) | |

*n/N-represents unknown bases
Splice site (/)
Exon refers to coding exons

The limits of the JAM2 cDNA sequence shown in FIG. 2 spans some 74,853 bp of genomic DNA. Various exons were also found in AP000223 (coding exon 1), AP000225 (coding exons 2, 3, 4, 5 and 6) and AP000226 (coding exons 6, 7, 8, 9 and 10). Since the complete JAM2 transcript(s) is considerably larger than 897 bp (FIG. 4), further exons in the untranslated regions remain to be identified either up and/or downstream. All intron/exon boundaries conform to the consensus CT/AG rule (see Breathnach, R., et al., Annu. Rev. Biochem., 50:359 (1981)).

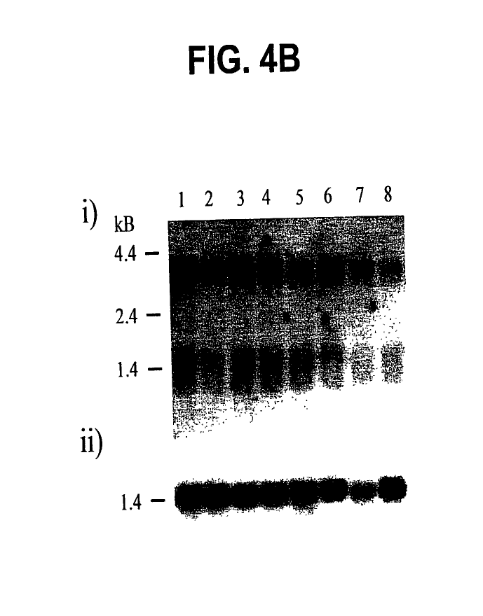

A mouse polyclonal serum was raised against the ectodomain of JAM2 in order to study protein expression and localization. The antibody was not useful for studying endogenous levels of JAM2 in native tissues. To gain further insight, a stable CHO cell line over-expressing JAM2 was generated. Using these cells detection of JAM2 protein by Western blot analysis was possible. FIG. 5 estimates the molecular mass of JAM2 to be 48 kDa. This is some 14 kDa larger than the size predicted from the peptide sequence. Glycosylation of JAM2 on at least one of its three N-lined glycosylation consensus sites could explain this phenomenon.

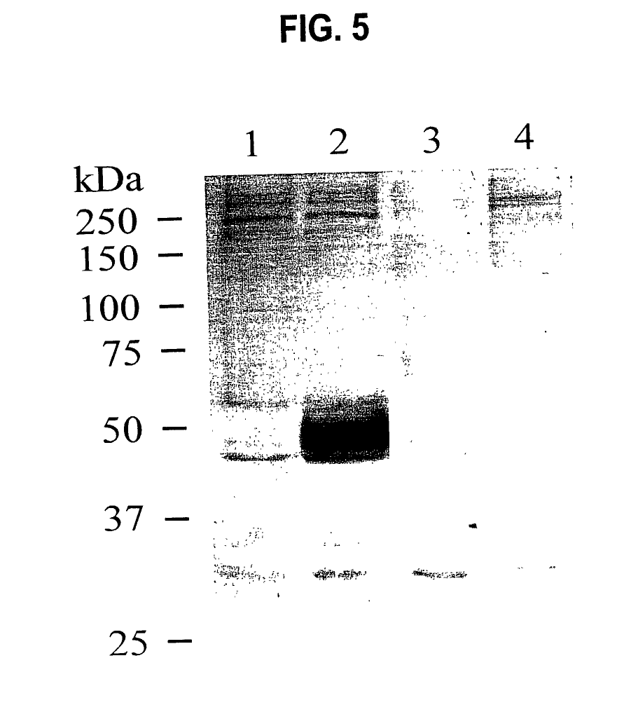

The CHO stable cell line was also used to determine cellular localization on confluent monolayers. FIG. 6 shows that JAM2 partitions to both surface membranes in addition to sites of cell-cell contact. The border pattern of staining is identical to that shown by mouse JAM (JAM1) expressed in CHO cells and endogenous human JAM (JAM1) in HUVECs (see, Martin-Padura, I., et al., J. Cell Biol. 142:117 (1998)).

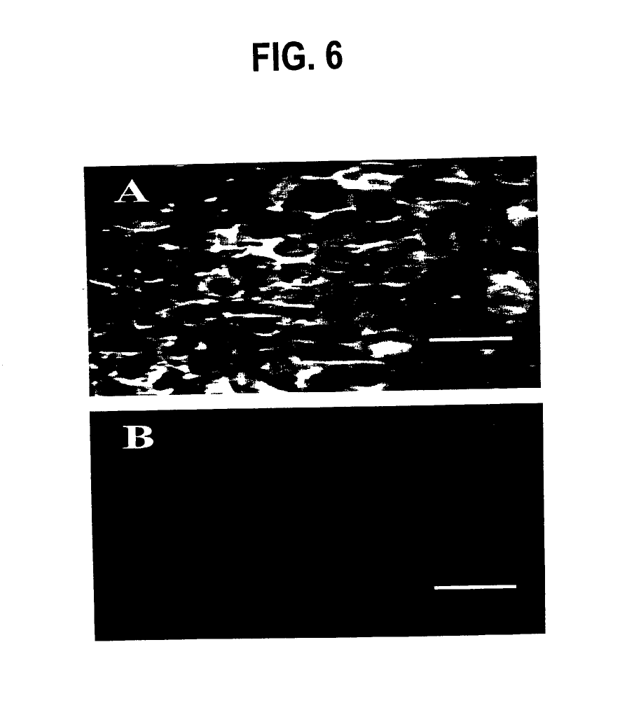

The capacity of JAM2 extracellular domain to adhere to various leukocyte cell lines according to a previously established in vitro binding assay performed under static conditions was next examined (see Todderud, G., J. Leukoc, Biol. 52:85(1992)). Calcein loaded cells were allowed to interact with JAM2-Fc captured in 96 well plates in binding buffer (hereinafter “BB”) which contained TBS plus 1 mM calcium, magnesium and manganese. Non-specific binding of cells to captured mIgG2a was determined simultaneously and subtracted. FIG. 7 shows that JAM2-Fc is able to capture the T lymphocyte cell lines HSB and HPB-ALL quite efficiently compared to interactions with B lymphocytes (RAMOS) and the monocytic cells HL60 and THP-1. Binding to the erythroleukemic K562 cell lines was non-existent.

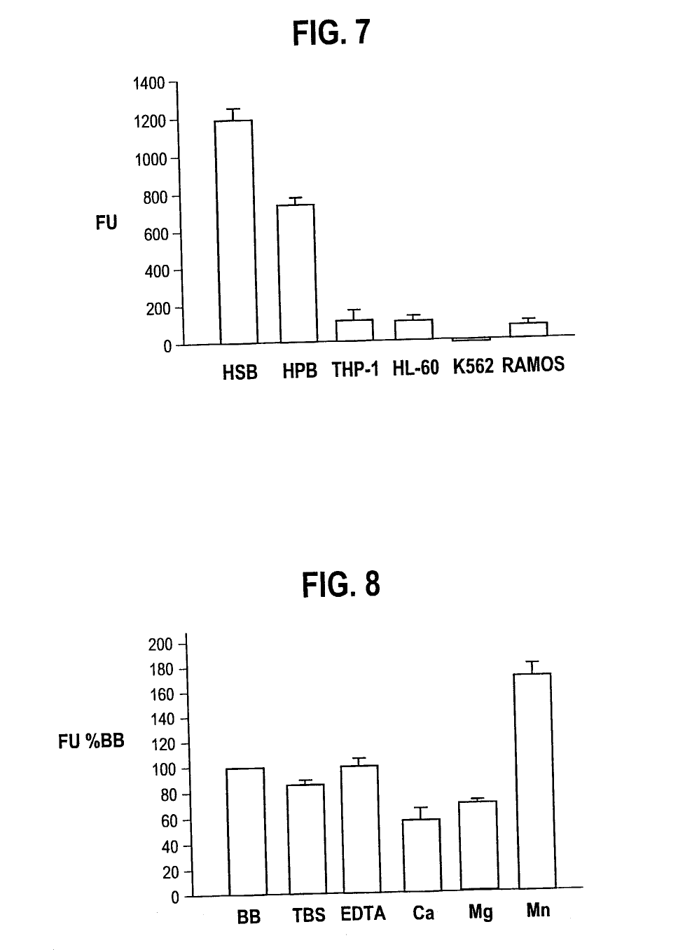

To further characterize the adhesion, the cation independence was investigated. Buffers were modified such that binding was performed in the presence of no cations or calcium, magnesium or manganese alone (see FIG. 8). There are two components to the adhesion. Firstly, a cation-independent interaction is demonstrated by the fact that EDTA does not inhibit binding below that obtained in the presence of all three cations. Secondly, a cation dependent interaction is described by a manganese specific enhancement of binding above that obtained in TBS or TBS+EDTA. This latter suggests integrin involvement. Since the screen conducted in FIG. 7 was performed under conditions favourable for cation-independent binding, all cell interactions in TBS plus manganese were reanalyzed. Manganese enhanced binding was not apparent on any of the other cell types.

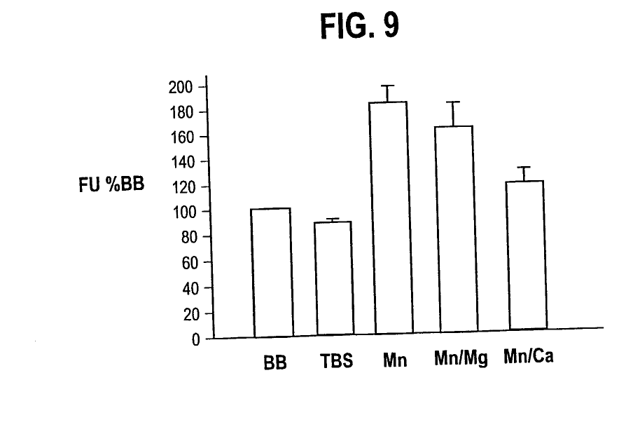

The JAM2/HSB manganese stimulated binding component is vitually abolished in the presence of calcium and magnesium (for example, in binding buffer). In order to determine if only one or both of these cations were inhibitory to the manganese augmentation, assays were performed using various cation combinations (see FIG. 9). The data show that inclusion of calcium in the manganese only buffer reduced interactions considerably (p<0.001). The effect of magnesium was statistically insignificant.

Mouse JAM (JAM1) is capable of homotypic interactions. Thus, it was examined whether JAM2 ectodomain bound HSB cells through this mechanism. FIG. 4 shows that, unlike human JAM (JAM1), JAM2 does not show expression in peripheral blood leukocytes. Nevertheless, to verify lack of expression in HSB cells, the mouse polyclonal serum was used to probe for JAM2 protein expression by Western blotting. No protein was detected (FIG. 5). As further proof, the surface JAM2 expression level was compared using the following more sensitive test. The HSB, control and JAM2 expressing CHO cells were loaded with calcein and incubated with either NMS or anti-JAM2 serum. Cell surface bound JAM2 antibody was detected by cell capture in 96 well plates coated with goat anti-mouse secondary antibodies. Table 3 shows that whilst the anti-JAM2 serum was very effective at capturing CHO cells expressing the JAM2 protein, no HSB cell binding was apparent.

TABLE 3

| Cell Type | Antibody | Av ± SEM |
|---|---|---|
| HSB | pre | 1,877 ± 234 |
| | JAM2 | 1,135 ± 97 |
| CHO control | pre | 1,210 ± 63 |
| | JAM2 | 1,019 ± 44 |
| CHO JAM2 | pre | 2,151 ± 287 |
| | JAM2 | 112,329 ± 4457 |

To extend these studies, the ability of the mouse anti-JAM2 serum to neutralize HSB binding to recombinant JAM2 was tested. Antibody was used to block epitopes on recombinant JAM2 captured on 96 well plates. Table 4 shows that whilst preimmune serum is ineffective, anti-JAM2 serum successfully prevents HSB binding. Since relatively high levels of JAM2 are coated on these wells, we were confident that if low levels were expressed on HSB cells, the antibody should be capable of producing inhibition when incubated directly with HSB cells. As predicted, under this experimental set-up, the anti-JAM2 antibody is unable to inhibit HSB interactions with recombinant JAM2.

TABLE 4

| Antibody | AV ± SEM |
|---|---|
| A) Preincubation with captured JAM2-Fc | |
| Preimmune | 1221 ± 54 |
| anti-JAM2 | 5 ± 2 |
| B) Preincubation with HSB cells | |
| Preimmune | 950 ± 45 |
| anti-JAM2 | 1138 ± 33 |

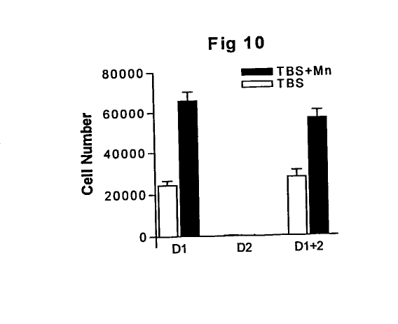

Many adhesion proteins belonging to the Ig superfamily utilize the most N-terminal Ig domain to achieve adhesion. To assess the binding capacity of the first Ig domain of JAM2, it was synthesized as a secreted protein in insect cells and binding compared with the full extracellular domain. FIG. 10 shows that this N-terminal Ig-fold of JAM2 is indeed capable of adhering to HSB cells. Further, the enhancement of binding in the presence of manganese was also retained.

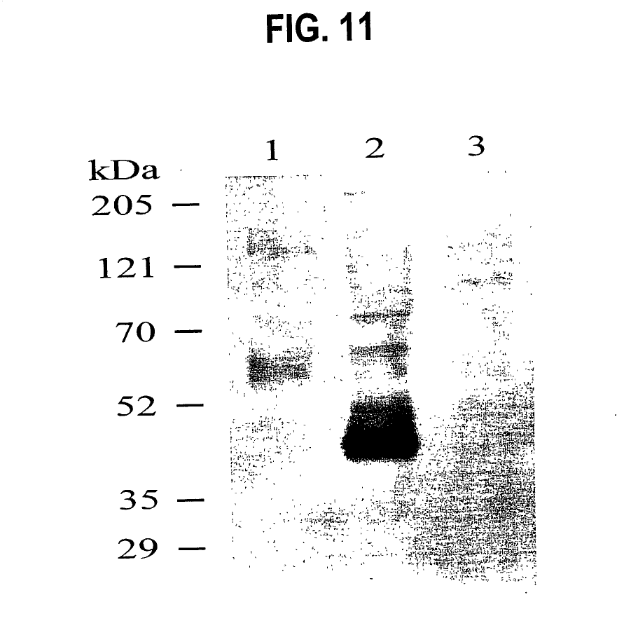

The inventors postulate that HSB cells express a counter-receptor for JAM2. To strengthen this hypothesis, and gain a preliminary characterization of the protein, the inventors performed precipitation experiments using JAM2-Fc. HSB cells were surface biotinylated, washed, lysed and incubated with JAM2-Fc in binding buffer. Bound proteins were precipitated using protein A and viewed on Western blots with avidin-HRP. FIG. 11 reveals that indeed JAM2 can specifically capture a surface protein from HSB cells of approximately 43 kDa. This band is not apparent in surface biotinylated K562 cells, in agreement with the cell adhesion studies described above. Further, human JAM1-Fc, which is unable to bind calcein loaded HSB cells and have characterized it as JAM3 (Arrate M P, Rodriguez J M, Tran T M, Brock T A, Cunningham S A, (2001) J. Biol. Chem. 276:45826-45832)

### EXAMPLE 3

### Identification of Compounds and Proteins That Modulate or Interfere with JAM2-α4 Integrin Binding.

JAM2-Fc adhesion to T cell lines was performed by capture of fusion protein by goat anti-mouse coated 96 well plates as described in Cunningham et al., J. Biol. Chem., 275:34750-34756 (2000). The HSB cells were loaded with calcien-AM (Molecular Probes Inc., Eugene Oreg.) and re-suspended in Tris Buffered Saline (TBS) with or without the addition of 1 mM MnCl₂. Binding to mouse IgG2a was used to subtract background. Soluble JAM3 was generated from a JAM3-Fc fusion that was digested with thrombin and purified to remove the Fc component. Neutralizing anti-JAM3 polyclonal serum was generated in mice using human JAM3 extracellular domain as immunogen. TBC 772 is a cyclic hexapeptide (C*WLDVC*) and known antagonist of alpha 4 integrins; TBC 1194 is a control scrambled peptide (C*DLVWC*) (Vanderslice, P., J. Immunol., 158:1710-1718 (1997)). For candidate drug additions, TBC 772 were added to the cells just prior to their addition to JAM2-Fc. Following adhesion for 90 minutes at 37° C., wells were washed 3 times at room temperature. Adhered cells were lyzed and fluorescence quantified in a Cytofluor with excitation at 485/20 nm and emission at 530/25.

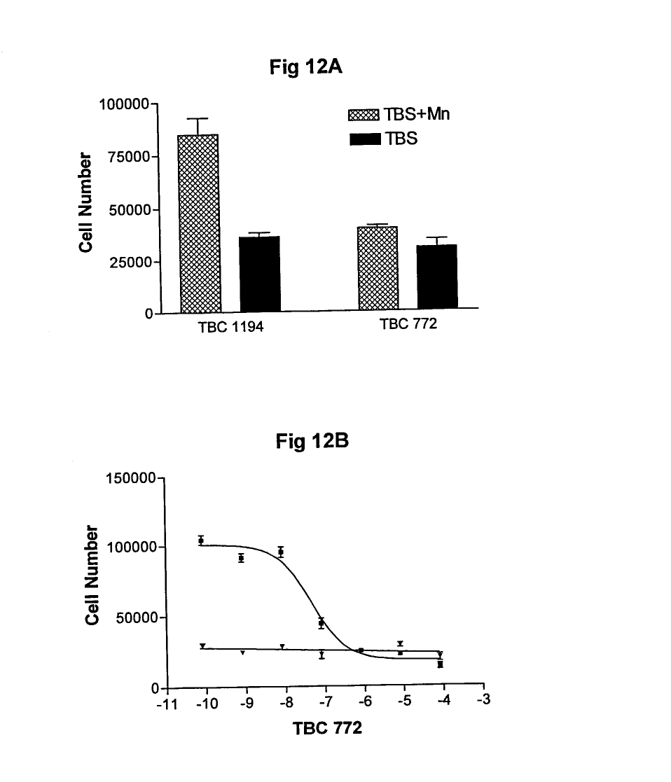

FIG. 12A shows that TBC 772 attenuates the manganese enhanced JAM2 adhesion to HSB cells. The cation-independent JAM2 interaction with JAM3 is not affected. TBC 1194 maintains both cation-independent and Mn-enhanced components. Based upon this data, the inventors believe that JAM2 interacts with both JAM3 and α4 integrins on cells which express α4 integrins, including, but not limited to, T cells.

FIG. 12B shows an example of the dose-response effect for TBC 772. While no effect is seen on the JAM2 interaction with JAM3 at any dose, the Mn-enhanced adhesion is progressively attenuated. The IC50 approximates to 60 nM.

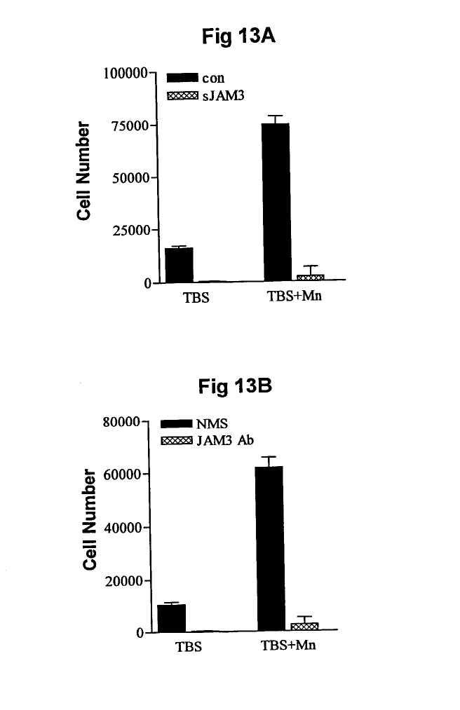

FIG. 13A shows that soluble JAM3 inhibits both the JAM2 interaction with JAM3 (TBS component), in addition to the further binding of JAM2 with α4 integrin (TBS+Mn component). The inventors believe that soluble JAM3 ectodomain would bind to the JAM2 on the well surface and prevent its interaction with JAM3 on the HSB cell surface. It is concluded that under these conditions, that JAM2 is unable to engage α4 integrin. Therefore, a specific interaction of JAM2 with cell-surface bound JAM3 is necessary before JAM2 can bind α4 integrin. Likewise, if neutralizing anti-JAM3 antibodies are used to prevent JAM2 binding to cell surface JAM3, then JAM2 is also unable to engage integrin (FIG. 13B).

Based upon this data, the inventors believe that JAM2 interacts with both JAM3 and α4 integrin on cells, including but not limited to T cells. Engagement of JAM2 with α4 is dependent upon JAM3 which suggests that JAM3 acts as a co-factor for JAM2 binding with integrin.

### EXAMPLE 4

### Identification of Specific Integrins that Adhere to JAM2

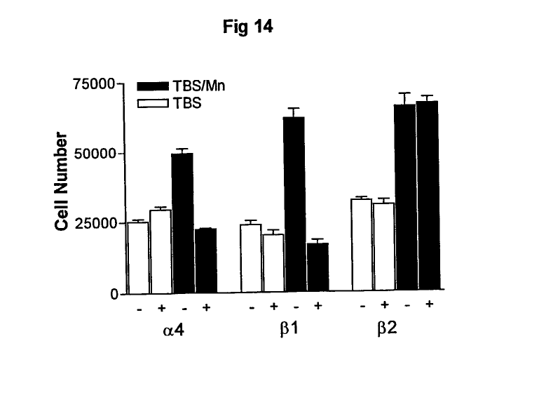

JAM2-Fc adhesion to HSB T cells was performed exactly as described above. Neutralizing integrin antibodies against either the α4, β1 or β2 subunits were added to the reaction. FIG. 14 shows that while both the α4 and β1 antibodies are effective inhibitors of the Mn-enhanced component of the JAM2 adhesion to HSB, the neutralizing β2 antibody is without effect. As expected, none of the antibodies affect the JAM2 binding to JAM3.

Based upon this data, the inventors conclude that JAM2 specifically interacts with the α4β1 integrin, and that JAM3 interacts with JAM2 to enhance the JAM2-α4β1 integrin interaction.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made to the composition, operation and arrangement of the method of the present invention described herein without departing from the concept and scope of the invention as defined in the following claims.

What is claimed is:

1. A method for identifying a compound that modulates binding between α4β1 or α4β7 integrin and junctional adhesion molecule 2 (JAM2), the method comprising the steps of:
contacting α4β1 or α4β7 integrin and JAM2 in the presence and absence of a test compound;
detecting binding between the α4β1 or α4β7 integrin and JAM2; and
identifying whether the compound modulates the binding between the α4β1 or α4β7 integrin and JAM2 in view of decreased or increased binding between the α4β1 or α4β7 integrin and JAM2 in the presence of the compound as compared to binding in the absence of the compound.

2. A method for identifying a compound that modulates binding between α4β1 or α4β7 integrin and junctional adhesion molecule 2 (JAM2), the method comprising the steps of:
a) immobilizing α4β1 or α4β7 integrin or a fusion protein or fragment thereof on a solid support to form an immobilized binding partner;
b) labeling JAM2 or a fusion protein or fragment thereof with a detectable agent to form a non-immobilized binding partner;
c) contacting the immobilized binding partner with the labeled non-immobilized binding partner in the presence and absence of a compound capable of specifically reacting with α4β1 or α4β7 integrin or JAM2;
d) detecting binding between the immobilized binding partner and the labeled non-immobilized binding partner; and
e) identifying compounds that affect binding between the immobilized binding partner and the labeled non-immobilized binding partner.

3. The method of claim 2 wherein the immobilized binding partner is contacted with the labeled non-immobilized binding partner in the presence of JAM3 and in the presence and absence of a compound capable of specifically reacting with α4β1 or α4β7 integrin or JAM2.

4. A method for identifying a compound that modulates binding between α4β1 or α4β7 integrin and junctional adhesion molecule 2 (JAM2), the method comprising the steps of:
a) immobilizing JAM2 or a fusion protein or fragment thereof on a solid support to form an immobilized binding partner;
b) labeling α4β1 or α4β7 integrin or a fusion protein or fragment thereof with a detectable agent to form a non-immobilized binding partner;
c) contacting the immobilized binding partner with the labeled non-immobilized binding partner in the presence and absence of a compound capable of specifically reacting with α4β1 or α4β7 integrin or JAM2;
d) detecting binding between the immobilized binding partner and the labeled non-immobilized binding partner; and
e) identifying compounds that affect binding between the immobilized binding partner and the labeled non-immobilized binding partner.

5. The method of claim 4 wherein the immobilized binding partner is contacted with the labeled non-immobilized binding partner in the presence of JAM3 and in the presence and absence of a compound capable of specifically reacting with α4β1 or α4β7 integrin or JAM2.

6. A method of inhibiting JAM2-α4β1 or α4β7 integrin mediated interactions in a mammal, the method comprising the step of administering to a mammal an effective amount of soluble JAM3 or soluble JAM2 to inhibit said interaction.

7. The method of claim 6 wherein the soluble JAM3 or soluble JAM2 is in recombinant form.

8. The method of claim 6 wherein soluble JAM3 or soluble JAM2 is a fusion protein or a full length extracellular JAM3 or JAM2 or fragment thereof.

9. A method of inhibiting JAM2-α4β1 or α4β7 integrin mediated interactions in a reaction mixture in vitro, the method comprising the step of adding an effective amount of soluble JAM3 or soluble JAM2 to the reaction mixture to inhibit said interaction.

10. The method of claim 9 wherein the soluble JAM3 or soluble JAM2 is in recombinant form.

11. The method of claim 10 wherein soluble JAM3 or soluble JAM2 is a fusion protein or a full length extracellular JAM3 or JAM2 or fragment thereof.

12. A method of preventing JAM2 mediated interaction with an alpha4 integrin in a mammal, the method comprising the step of administering to said mammal an effective amount of a compound identified pursuant to claims 1, 2 or 4 to prevent said interaction.

13. A method of preventing JAM2 mediated interaction with an α4β1 or α4β7 integrin in a reaction mixture in vitro, the method comprising the step of administering to said reaction mixture an effective amount of a compound identified pursuant to claims 1, 2 or 4 to prevent said interaction.
